(19) United States
(12) Patent Application Publication
(10) Pub. No.: US 2022/0363776 A1
(43) Pub. Date: Nov. 17, 2022

(54) METHODS AND PHARMACEUTICAL COMPOSITION FOR THE TREATMENT OF OVARIAN CANCER, BREAST CANCER OR PANCREATIC CANCER

(71) Applicants: INSERM (INSTITUT NATIONAL DE LA SANTÉ ET DE LA RECHERCHE MÉDICALE), Paris (FR); CENTRE NATIONAL DE LA RECHERCHE SCIENTIFIQUE (CNRS), Paris (FR); UNIVERSITÉ D'AIX MARSEILLE, Marseille Cedex (FR); AARHUS UNIVERSITET, Aarhus C (DK)

(72) Inventors: Anders Etzerodt, Hinnerup (DK); Toby Lawrence, London (GB)

(21) Appl. No.: 17/754,403

(22) PCT Filed: Oct. 2, 2020
(86) PCT No.: PCT/EP2020/077677

§ 371 Date: Mar. 31, 2022

(30) Foreign Application Priority Data
Oct. 4, 2019 (EP) 19306281.7

(51) Int. Cl. C07K 16/28; A61P 35/00

(52) U.S. Cl. CPC C07K 16/2896; A61P 35/00

(57) ABSTRACT

Experimental and clinical evidence suggests tumor-associated macrophages (TAM) play important roles in cancer progression. Here, the inventors show that the omentum is a critical pre-metastatic niche for development of invasive disease in this model and defined a unique subset of CD163+ Tim4+ tissue-resident macrophages in omentum of embryonic origin and maintained independently of bone marrow-derived monocytes. Transcriptomic analysis showed that resident CD163+ Tim4+ omental macrophages were phenotypically distinct and maintained their resident identity during tumor growth. Selective depletion of CD163+ Tim4+ macrophages in omentum using genetic and therapeutic tools prevented tumor progression and metastatic spread of disease. The molecular pathways of cross-talk between tissue-resident macrophages and disseminated cancer cells may represent new targets to prevent metastasis and disease recurrence. Thus the present invention relates to a method of treating ovarian cancer, breast cancer and pancreatic cancer in a subject in need thereof comprising administering to the subject a therapeutically effective of an agent capable of depleting the population of CD163+ Tim4+ tumor associated macrophages.

### FIELD OF THE INVENTION

The present invention relates to methods and pharmaceutical composition for the treatment of ovarian cancer, breast cancer or pancreatic cancer.

### BACKGROUND OF THE INVENTION

Macrophages populate all human tissues and their involvement in tumor progression and metastasis are well documented (Noy and Pollard, 2014). Recent advances in our understanding of macrophage biology suggest that tissue-resident macrophages and infiltrating tumor-associated macrophages (TAM) display a high degree of heterogenity, both in terms of phenotype and ontogeny. However, our understanding of the physiological relevance of this heterogeneity and its implications for tumor development is still limited. In particular, the role of resident macrophages in tissue-specific tumor initiation and progression is unclear.

Ovarian cancer is the 8th leading cause of cancer-related death in women worldwide and has a particularly poor prognosis due to almost 80% of cases being diagnosed with late-stage invasive disease (Ferlay et al., 2018). High-grade serous ovarian carcinoma (HGSOC), the most frequent and aggressive form of ovarian cancer, is characterized by the formation of malignant ascites and peritoneal metastases which results in a particularly disastrous prognosis (Lengyel, 2010). HGSOC originates from transformation of fallopian tube or ovarian surface epithelial cells that can disseminate at early stages into the peritoneal cavity by exfoliation (Lengyel, 2010). Due to the lack of any anatomical barriers, exfoliated cancer cells are carried by the peritoneal fluid and spread throughout abdominal cavity in a process termed transcolemic metastasis (Kipps et al., 2013). Several reports have also suggested that ovarian cancer cells in ascites acquire cancer stem cell (CSC)-like properties that play particularly important roles in metastatic spread, chemosensitivity and disease recurrence post therapy (Bapat et al., 2005). The most frequent site for metastasis in HGSOC is the omentum (Sehouli et al., 2009), an apron of visceral adipose tissue in the abdomen formed from a fold of the peritoneal mesothelium. Omentum contains a high density of lymphoid aggregates known as milky spots or fat-associated lymphoid clusters (FALC), which are thought to contribute to peritoneal and intestinal immunity (Bénézech et al., 2015; Krist et al., 1995; Rangel-Moreno et al., 2009). The tropism of ovarian cancer cells for the omentum and the implications for disease progression in HGSOC are not yet fully understood. Several reports have suggested that FALCs play an active role in colonization of omentum (Hagiwara et al., 1993), but the tumor-promoting function of FALCs was shown to be independent of both B and T lymphocytes (Clark et al., 2013). Myeloid cells are also abundant in FALCs and macrophage density was recently shown to increase proportionally with disease score in omenta from ovarian cancer patients (Pearce et al., 2018). However, the specific role of omental macrophages in colonization and disease progression remains to be explored.

Resident macrophages populate all tissues and perform trophic functions that contribute to organ development, tissue remodeling and homeostasis (Pollard, 2009). Experimental evidence has shown that TAM contribute to tumor progression by promoting angiogenesis, matrix remodeling and epithelial to mesenchymal transition (EMT) (Raggi et al., 2015), which ultimately leads to increased cell invasion and metastasis (Noy and Pollard, 2014). These properties reflect the trophic functions of tissue-resident macrophages in development, and consistent with these developmental functions the transcriptome of TAM from mammary gland tumors has been shown to be enriched for genes that also define embryonic macrophages (Ojalvo et al., 2010). Until recently, it was thought that tissue macrophages were maintained from bone marrow-derived monocyte precursors, with the exception of the brain microglia and Langerhans' cells in the skin (Geissmann et al., 2010), and the same has generally been assumed for TAM (Lahmar et al., 2016a). However, recent advances in molecular techniques that allow the fate-mapping of macrophages in vivo have revealed that most tissue macrophages originate from embryonic precursors and can be maintained by local proliferation with little input from the bone marrow (Schulz et al., 2012). Subsequent studies have shown that tissue-resident macrophages of embryonic origin can be gradually replaced by monocyte-derived cells to varying degrees depending on the specific tissue (Ginhoux and Guilliams, 2016). The functional implications of these distinct developmental origins and their respective contributions to tumorigenesis has not yet been fully explored. However, recent reports suggest that embryo-derived tissue-resident macrophages can proliferate during tumor development and have distinct functions in tumor progression compared to monocyte-derived TAM (Loyher et al., 2018; Zhu et al., 2017). Therefore, it is critically important to further our understanding of the specific axes of cross-talk between tissue-resident macrophages and cancer cells, both during tumor initiation and the malignant progression of disseminated tumor cells, as this may reveal new targets to combat tumor progression and thwart the development of invasive disease.

Here we show that omentum represents a critical pre-metastatic niche in a mouse model of metastatic ovarian cancer. We further defined a specific subset of tissue-resident macrophages in omentum that express CD163 and Tim4 that are required for the metastatic spread of ovarian cancer cells. Using genetic fate-mapping and shielded chimera experiments, we show that CD163+ Tim4+ omental macrophages are derived from embryonic progenitors and maintained independently of bone marrow-derived monocytes. Specific depletion of CD163+ Tim4+ macrophages using genetic tools or antibody-targeted cytotoxic liposomes, was sufficient to prevent the development of metastatic disease, whereas depletion of monocyte-derived TAM had no impact. These studies suggest that specific interactions between disseminated tumor cells and tissue-resident CD163+ Tim4+ macrophages in the omentum promotes the malignant progression of ovarian cancer. The molecular pathways that underly these interactions may represent new therapeutic targets for treatment of invasive metastatic disease.

### SUMMARY OF THE INVENTION

The present invention relates to methods and pharmaceutical composition for the treatment of ovarian cancer, breast cancer or pancreatic cancer. In particular, the present invention is defined by the claims.

### DETAILED DESCRIPTION OF THE INVENTION

Experimental and clinical evidence suggests tumor-associated macrophages (TAM) play important roles in cancer progression. Here, the inventors have characterized the ontogeny and function of TAM subsets in a mouse model of metastatic ovarian cancer that is representative for visceral peritoneal metastasis. They show that the omentum is a critical pre-metastatic niche for development of invasive disease in this model and defined a unique subset of CD163+ Tim4+ tissue-resident macrophages in omentum of embryonic origin and maintained independently of bone marrow-derived monocytes. Transcriptomic analysis showed that resident CD163+ Tim4+ omental macrophages were phenotypically distinct and maintained their resident identity during tumor growth. Selective depletion of CD163+ Tim4+ macrophages in omentum using genetic and therapeutic tools prevented tumor progression and metastatic spread of disease. These studies describe a specific role for tissue-resident macrophages in the invasive progression of metastatic ovarian cancer. The molecular pathways of cross-talk between tissue-resident macrophages and disseminated cancer cells may represent new targets to prevent metastatsis and disease recurrance.

As used herein, the term “CD163” (Cluster of Differentiation 163) also known as M130 MM130 or “SCARI1 has its general meaning in the art and refers to a protein that in humans is encoded by the CD163 gene [Gene ID: 9332]. CD163 is exclusively expressed in monocytes and macrophages. It functions as an acute phase-regulated receptor involved in the clearance and endocytosis of hemoglobin/haptoglobin complexes by macrophages, and may thereby protect tissues from free hemoglobin-mediated oxidative damage. This protein may also function as an innate immune sensor for bacteria and inducer of local inflammation. The molecular size is 130 kDa. The receptor belongs to the scavenger receptor cysteine rich family type B and consists of a 1048 amino acid residues extracellular domain, a single transmembrane segment and a cytoplasmic tail with several splice variants. An exemplary human amino acid sequence is represented by SEQ ID NO:1. The extracellular domain of CD163 ranges from the amino acid residue at position 42 to the amino acid residue 1050 at position in SEQ ID NO:1.

SEQ ID NO: 1>sp|Q86VB7|C163A_HUMAN Scavenger
receptor cysteine-rich type 1 protein M130
OS = Homo sapiens OX = 9606 GN = CD163 PE = 1
SV = 2. The extracellular domains is shown as
underlined.
MSKLRMVLLEDSGSADFRRHFVNLSPFTITVVLLLSACFVTSSLGGTDK
ELRLVDGENKCSGRVEVKVQEEWGTVCNNGWSMEAVSVICNQLGCPTAI
KAPGWANSSAGSGRIWMDHVSCRGNESALWDCKHDGWGKHSNCTHQQDA
GVTCSDGSNLEMRLTRGGNMCSGRIEIKFQGRWGTVCDDNFNIDHASVI
CRQLECGSAVSFSGSSNFGEGSGPIWFDDLICNGNESALWNCKHQGWGK
HNCDHAEDAGVICSKGADLSLRLVDGVTECSGRLEVRFQGEWGTICDDG
WDSYDAAVACKQLGCPTAVTAIGRVNASKGFGHIWLDSVSCQGHEPAIW
QCKHHEWGKHYCNHNEDAGVTCSDGSDLELRLRGGGSRCAGTVEVEIQR
LLGKVCDRGWGLKEADVVCRQLGCGSALKTSYQVYSKIQATNTWLFLSS
CNGNETSLWDCKNWQWGGLTCDHYEEAKITCSAHREPRLVGGDIPCSGR
VEVKHGDTWGSICDSDFSLEAASVLCRELQCGTVVSILGGAHFGEGNGQ
IWAEEFQCEGHESHLSLCPVAPRPEGTCSHSRDVGVVCSRYTEIRLVNG
KTPCEGRVELKTLGAWGSLCNSHWDIEDAHVLCQQLKCGVALSTPGGAR
FGKGNGQIWRHMFHCTGTEQHMGDCPVTALGASLCPSEQVASVICSGNQ
SQTLSSCNSSSLGPTRPTIPEESAVACIESGQLRLVNGGGRCAGRVEIY
HEGSWGTICDDSWDLSDAHVVCRQLGCGEAINATGSAHFGEGTGPIWLD
EMKCNGKESRIWQCHSHGWGQQNCRHKEDAGVICSEFMSLRLTSEASRE
ACAGRLEVFYNGAWGTVGKSSMSETTVGVVCRQLGCADKGKINPASLDK
AMSIPMWVDNVQCPKGPDTLWQCPSSPWEKRLASPSEETWITCDNKIRL
QEGPTSCSGRVEIWHGGSWGTVCDDSWDLDDAQVVCQQLGCGPALKAFK
EAEFGQGTGPIWLNEVKCKGNESSLWDCPARRWGHSECGHKEDAAVNCT
DISVQKTPQKATTGRSSRQSSFIAVGILGVVLLAIFVALFFLTKKRRQR
QRLAVSSRGENLVHQIQYREMNSCLNADDLDLMNSSENSHESADFSAAE
LISVSKFLPISGMEKEAILSHTEKENGNL

As used herein, the term “Tim4” (T-cell immunoglobulin and mucin domain containing 4) also known as TIMD-4 has its general meaning in the art and refers to a protein that in humans is encoded by the TIMD4 gene located on chromosome 5q33.2 [Gene ID: 91937]. Tim4 contains IgV domain with integrin-binding site as well as a unique metal-ion-dependent ligand binding site for phosphatidylserin. Unlike other TIMs that are mainly expressed on T cells, Tim4 is expressed on APCs such as dendritic cells or macrophages. Tim4 serves as a ligand for TIM-1 but also as a receptor for phosphatidylserin. Its phosphatidylserin binding properties also play an important role in removal of apoptotic cells. Tim4 expression on macrophages plays an important role in their homeostatic maintenance. An exemplary human amino acid sequence is represented by SEQ ID NO:2. The extracellular domain of Tim4 ranges from the amino acid residue at position 25 to the amino acid residue 314 at position in SEQ ID NO:2.

SEQ ID NO: 2>sp|Q96H15|TIMD4_HUMAN T-cell immuno-
globulin and mucin domain-containing protein 4
TIM4 OS = Homo sapiens OX = 9606 GN = TIMD4.
The extracellular domains is shown as underlined.
Mskeplilwlmiefwwlyltpvtsetvvtevlghrvtlpclysswshns
nsmcwgkdqcpysgckealirtdgmrvtsrksakyrlqgtiprgdvslt
ilnpsesdsgvyccrievpgwfndvkinvrlnlqrasttthrtattttr
rttttsptttrqmtttpaalpttvvttpdlttgtplqmttiavfttant
clsltpstlpeeatglltpepskegpiltaesetvlpsdswssvestsa
dtvlltskeskvwdlpstshvsmwktsdsvsspqpgasdtavpeqnktt
ktgqmdgipmsmknempisqllmiiapslgfvlfalfvafllrgklmet
ycsqkhtrldyigdsknvlndvqhgrededglftl

As used herein, the term “tumor associated macrophage” or “TAM” has its general meaning in the art and is intended to describe a type of cell belonging to the macrophage lineage. They are found in close proximity or within tumor masses. TAMs are derived from circulating monocytes or resident tissue macrophages, which form the major leukocytic infiltrate found within the stroma of many tumor types. Accordingly, the term “CD163+ Tim4+ tumor associated macrophages” refers to a subset of TAM characterized by the expression of CD163 and Tim4+.

As used herein, the term “tissue-resident macrophages” has its general meaning in the arts and refers to a heterogeneous population of immune cells that fulfill tissue-specific and niche-specific functions. These range from dedicated homeostatic functions, such as clearance of cellular debris and iron processing, to central roles in tissue immune surveillance, response to infection and the resolution of inflammation.

As used herein, the term “omentum” has its general meaning in the arts and refers to layers of peritoneum that surround abdominal organs, such as the stomach and liver. The omentum form an apron of visceral adipose tissue in the abdomen. Omentum contains a high density of lymphoid aggregates known as milky spots or fat-associated lymphoid clusters (FALC), which are thought to contribute to peritoneal and intestinal immunity.

Accordingly, the first object of the present invention relates to a method for treating cancer selected in the group consisting of ovarian cancer, breast cancer and pancreatic cancer in a subject in need thereof comprising administering to the subject a therapeutically effective amount of an agent capable of depleting the population of CD163+ Tim4+ macrophages.

In other word, the present invention relates to an agent capable of depleting the population of CD163+ Tim4+ macrophages for use in the treatment of cancer selected in the group consisting of ovarian cancer, breast cancer or pancreatic cancer in a subject in need thereof.

In some embodiment, the cancer is an ovarian cancer.

Thus, the invention refers to a method for treating ovarian cancer in a subject in need thereof comprising administering to the subject a therapeutically effective amount of an agent capable of depleting the population of CD163+ Tim4+ macrophages.

In some embodiment, the agent is capable of depleting the population of CD163+ Tim4+ macrophages in omentum.

In some embodiment, the CD163+ Tim4+ macrophages are CD163+ Tim4+ tissue-resident macrophages.

In particular embodiment the CD163+ Tim4+ macrophages are tumor-associated macrophages (TAM).

As used herein, the term “ovarian cancer” has its general meaning in the art and refers to a cancer that forms in or on an ovary. Ovarian cancer include, but is not limited to: ovarian epithelial tumors such as ovarian mucinous carcinoma, high-grade serous carcinoma, ovarian endometrioid carcinoma, ovarian clear-cell carcinoma, ovarian low malignant potential tumors and primary peritoneal carcinoma; germ cell tumors such as teratomas, dysgerminoma ovarian germ cell cancer, choriocarcinoma tumors and endodermal sinus tumors; sex-cord stromal tumors such as granulosa cell tumors, granulosa-theca tumors, ovarian fibroma, leydic cell tumors, sertoli cell tumors, sertoli-leydig tumors and gynandroblastoma; ovarian sarcoma such as ovarian carcinosarcomas, ovarian adenosarcomas, ovarian leiomyo sarcomas and ovarian fibrosarcomas; krukenberg tumors; and ovarian cysts. The term “ovarian cancer” further encompasses both primary and metastatic ovarian cancers.

As used herein, the term “breast cancer” has its general meaning in the art and refers to a cancer that forms in the cells of the breasts. Breast cancer include basal breast cancer, metastatic breast cancer or triple negative breast cancer. As used herein the expression “Triple negative breast cancer” has its general meaning in the art and means that said breast cancer lacks receptors for the hormones estrogen (ER-negative) and progesterone (PR-negative), and for the protein HER2.

As used herein, the term “pancreatic cancer” has its general meaning in the art and refers to a cancer caused by the abnormal and uncontrolled growth of cells in the pancreas. Pancreatic cancer include pancreatic exocrine cancer such as adenocarcinoma, acinar cell carnoma, intraductal papillary-mucinus neoplasm and mucinous cystic neoplasm; and pancreatic neuroendocrine cancer such as gastrinoma, glucaganoma, insulinoma, somatostaninoma and non-functional islet cell tumor.

As used herein, the term “subject” refers to any mammal, such as rodent, a feline, a canine, a primate or human. In some embodiment of the invention, the subject refers to any subject afflicted with or susceptible to be afflicted with ovarian cancer, breast cancer or pancreatic cancer. Particularly, in preferred embodiment, the subject is a human afflicted with or susceptible to be afflicted with ovarian cancer.

In some embodiment, the subject is a human afflicted with or susceptible to be afflicted with ovarian cancer and who has developed an immune checkpoint therapy, chemotherapy or radiotherapy resistance.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “therapeutically effective amount” refers to an amount effective, at dosages and for periods of time necessary, to achieve a desired therapeutic result. A therapeutically effective amount of the active agent may vary according to factors such as the disease state, age, sex, and weight of the individual, and the ability of the active agent to elicit a desired response in the individual. A therapeutically effective amount is also one in which any toxic or detrimental effects of the antibody or antibody portion are outweighed by the therapeutically beneficial effects. The efficient dosages and dosage regimens for the active agent depend on the disease or condition to be treated and may be determined by the persons skilled in the art. A physician having ordinary skill in the art may readily determine and prescribe the effective amount of the pharmaceutical composition required. For example, the physician could start doses of active agent employed in the pharmaceutical composition at levels lower than that required achieving the desired therapeutic effect and gradually increasing the dosage until the desired effect is achieved. In general, a suitable dose of a composition of the present invention will be that amount of the compound, which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. Typically, the ability of a compound to inhibit cancer may, for example, be evaluated in an animal model system predictive of efficacy in human tumors. A therapeutically effective amount of a therapeutic compound may decrease tumor size, or otherwise ameliorate symptoms in a patient. One of ordinary skill in the art would be able to determine such amounts based on such factors as the patient's size, the severity of the patient's symptoms, and the particular composition or route of administration selected. An exemplary, non-limiting range for a therapeutically effective amount of a inhibitor of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of a inhibitor of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the methods of treatment and uses according to the invention are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans, for example using a labeled inhibitor of the present invention, fragment or mini-antibody derived from the inhibitor of the present invention. If desired, an effective daily dose of a pharmaceutical composition may be administered as two, three, four, five, six or more sub-doses administered separately at appropriate intervals throughout the day, optionally, in unit dosage forms. In some embodiments, the human monoclonal antibodies of the present invention are administered by slow continuous infusion over a long period, such as more than 24 hours, in order to minimize any unwanted side effects. An effective dose of a inhibitor of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of a inhibitor of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

As used herein the terms “administering” or “administration” refer to the act of injecting or otherwise physically delivering a substance as it exists outside the body (e.g., an inhibitor of IRE1α) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

The agent capable of depleting the population of CD163+ Tim4+ macrophages of the invention can be administered in combination with an agent capable of depleting the population of TAM that only express CD163.

Thus, the invention also refers to a method for treating cancer selected in the group consisting of ovarian cancer, breast cancer and pancreatic cancer in a subject in need thereof comprising administering to the subject a therapeutically effective combination of an agent capable of depleting the population of CD163+ Tim4+ macrophages and an agent capable of depleting the population of TAM that only express CD163.

The agent capable of depleting the population of CD163+ Tim4+ macrophages of the invention can be administered in combination with a classical treatment of cancer.

Thus, the invention also refers to a method for treating an ovarian cancer, breast cancer or pancreatic cancer in a subject in need thereof comprising administering to the subject a therapeutically effective combination of an agent capable of depleting the population of CD163+ Tim4+ macrophages and a classical treatment of cancer.

As used herein, the term “classical treatment” refers to any compound, natural or synthetic, used for the treatment of ovarian cancer, breast cancer or pancreatic cancer. In a particular embodiment, classical treatment refers to radiation therapy, immune checkpoint therapy or chemotherapy.

According to the invention, compound used for the classical treatment of ovarian cancer, breast cancer or pancreatic cancer may be selected in the group consisting in: EGFR inhibitor such as cetuximab, panitumumab, bevacizumab and ramucirumab; PARP inhibitors such as olaparib, rucaparib and niparib; immune checkpoint inhibitor; chemotherapeutic agent and radiotherapeutics agent.

As used herein, the term “chemotherapy” refers to cancer treatment that uses one or more chemotherapeutic agents.

As used herein, the term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a camptothecin (including the synthetic analogue topotecan and irinotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33: 183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, trifluridine, tipiracil, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®, razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum such as oxaliplatin, cisplatin and carbloplatin; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; ziv-aflibercept; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

As used herein, the term “radiation therapy” has its general meaning in the art and refers the treatment of colorectal cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a colorectal cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

As used herein, the term “immune checkpoint therapy” refers to cancer treatment that uses one or more immune checkpoint inhibitor.

As used herein, the term “immune checkpoint inhibitor” has its general meaning in the art and refers to any compound inhibiting the function of an immune inhibitory checkpoint protein. As used herein the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of inhibitory checkpoint molecules include A2AR, B7-H3, B7-H4, BTLA, CTLA-4, CD277, IDO, KIR, PD-1, LAG-3, TIM-3 and VISTA. Inhibition includes reduction of function and full blockade. Preferred immune checkpoint inhibitors are antibodies that specifically recognize immune checkpoint proteins. A number of immune checkpoint inhibitors are known and in analogy of these known immune checkpoint protein inhibitors, alternative immune checkpoint inhibitors may be developed in the (near) future. The immune checkpoint inhibitors include peptides, antibodies, nucleic acid molecules and small molecules. Examples of immune checkpoint inhibitor includes PD-1 antagonist, PD-L1 antagonist, PD-L2 antagonist CTLA-4 antagonist, VISTA antagonist, TIM-3 antagonist, LAG-3 antagonist, IDO antagonist, KIR2D antagonist, A2AR antagonist, B7-H3 antagonist, B7-H4 antagonist, and BTLA antagonist.

In some embodiments, PD-1 (Programmed Death-1) axis antagonists include PD-1 antagonist (for example anti-PD-1 antibody), PD-L1 (Programmed Death Ligand-1) antagonist (for example anti-PD-L1 antibody) and PD-L2 (Programmed Death Ligand-2) antagonist (for example anti-PD-L2 antibody). In some embodiments, the anti-PD-1 antibody is selected from the group consisting of MDX-1106 (also known as Nivolumab, MDX-1106-04, ONO-4538, BMS-936558, and Opdivo®), Merck 3475 (also known as Pembrolizumab, MK-3475, Lambrolizumab, Keytruda®, and SCH-900475), and CT-011 (also known as Pidilizumab, hBAT, and hBAT-1). In some embodiments, the PD-1 binding antagonist is AMP-224 (also known as B7-DCIg). In some embodiments, the anti-PD-L1 antibody is selected from the group consisting of YW243.55.S70, MPDL3280A, MDX-1105, and MEDI4736. MDX-1105, also known as BMS-936559, is an anti-PD-L1 antibody described in WO2007/005874. Antibody YW243.55. S70 is an anti-PD-L1 described in WO 2010/077634 A1 MEDI4736 is an anti-PD-L1 antibody described in WO2011/066389 and US2013/034559. MDX-1106, also known as MDX-1106-04, ONO-4538 or BMS-936558, is an anti-PD-1 antibody described in U.S. Pat. No. 8,008,449 and WO2006/121168. Merck 3745, also known as MK-3475 or SCH-900475, is an anti-PD-1 antibody described in U.S. Pat. No. 8,345,509 and WO2009/114335. CT-011 (Pidizilumab), also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342. Atezolimumab is an anti-PD-L1 antibody described in U.S. Pat. No. 8,217,149. Avelumab is an anti-PD-L1 antibody described in US 20140341917. CA-170 is a PD-1 antagonist described in WO2015033301 & WO2015033299. Other anti-PD-1 antibodies are disclosed in U.S. Pat. No. 8,609,089, US 2010028330, and/or US 20120114649. In some embodiments, the PD-1 inhibitor is an anti-PD-1 antibody chosen from Nivolumab, Pembrolizumab or Pidilizumab. In some embodiments, PD-L1 antagonist is selected from the group comprising of Avelumab, BMS-936559, CA-170, Durvalumab, MCLA-145, SP142, STI-A1011, STIA1012, STI-A1010, STI-A1014, A110, KY1003 and Atezolimumab and the preferred one is Avelumab, Durvalumab or Atezolimumab.

In some embodiments, CTLA-4 (Cytotoxic T-Lymphocyte Antigen-4) antagonists are selected from the group consisting of anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (Ipilimumab), Tremelimumab, anti-CD28 antibodies, anti-CTLA-4 adnectins, anti-CTLA-4 domain antibodies, single chain anti-CTLA-4 fragments, heavy chain anti-CTLA-4 fragments, light chain anti-CTLA-4 fragments, inhibitors of CTLA-4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B. Additional CTLA-4 antibodies are described in U.S. Pat. Nos. 5,811,097; 5,855,887; 6,051,227; and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17): 10067-10071 (1998); Camacho et al., J. Clin: Oncology, 22(145): Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281. A preferred clinical CTLA-4 antibody is human monoclonal antibody (also referred to as MDX-010 and Ipilimumab with CAS No. 477202-00-9 and available from Medarex, Inc., Bloomsbury, N.J.) is disclosed in WO 01/14424. With regard to CTLA-4 antagonist (antibodies), these are known and include Tremelimumab (CP-675,206) and Ipilimumab.

In some embodiments, the immune checkpoint therapy consists in administering to the patient a combination of a CTLA-4 antagonist and a PD-1 antagonist.

Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). Also included are TIM-3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94). As used herein, the term “TIM-3” has its general meaning in the art and refers to T cell immunoglobulin and mucin domain-containing molecule 3. The natural ligand of TIM-3 is galectin 9 (Ga19). Accordingly, the term “TIM-3 inhibitor” as used herein refers to a compound, substance or composition that can inhibit the function of TIM-3. For example, the inhibitor can inhibit the expression or activity of TIM-3, modulate or block the TIM-3 signaling pathway and/or block the binding of TIM-3 to galectin-9. Antibodies having specificity for TIM-3 are well known in the art and typically those described in WO2011155607, WO2013006490 and WO2010117057.

In some embodiments, the immune checkpoint inhibitor is an IDO inhibitor. Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. Preferably the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In some embodiment, the classical treatment consist in administering at least one immune checkpoint inhibitor.

As used herein the term “co-administering” as used herein means a process whereby the combination of the agent capable of depleting the population of CD163+ Tim4+ macrophages and the classical treatment, is administered to the same patient. The agent capable of depleting the population of CD163+ Tim4+ macrophages and the classical treatment may be administered simultaneously, at essentially the same time, or sequentially. If administration takes place sequentially, the agent capable of depleting the population of CD163+ Tim4+ macrophages is administered before the classical treatment. In some embodiment, the agent capable of depleting the population of CD163+ Tim4+ macrophages and the classical treatment need not be administered by means of the same vehicle. The agent capable of depleting the population of CD163+ Tim4+ macrophages and the classical treatment may be administered one or more times and the number of administrations of each component of the combination may be the same or different. In addition, the agent capable of depleting the population of CD163+ Tim4+ macrophages and the classical treatment need not be administered at the same site.

As used the terms “combination” and “combination therapy” are interchangeable and refer to treatments comprising the administration of at least two compounds administered simultaneously, separately or sequentially. As used herein the term “co-administering” as used herein means a process whereby the combination of at least two compounds is administered to the same patient. The at least two compounds may be administered simultaneously, at essentially the same time, or sequentially. The at least two compounds can be administered separately by means of different vehicles or composition. The at least two compounds can also administered in the same vehicle or composition (e.g. pharmaceutical composition). The at least two compounds may be administered one or more times and the number of administrations of each component of the combination may be the same or different.

The acquisition of stem-like characteristics by cancer cells (cancer stem cells; CSC) has been suggested to promote tumor progression and metastasis (Kreso and J. E. Dick, 2014). CSCs show increased anchorage-independent survival and high levels of resistance to chemotherapy or radiotherapy and thus have major implications for disease recurrence from disseminated tumor cells. Herein, the inventors demonstrate that CD163⁺ Tim4⁺ tissue-resident macrophages in omentum promote the CSC-like phenotype ovarian cancer cells.

Accordingly, a further object of the present invention relates to a method of treating cancer resistant to immune checkpoint therapy, chemotherapy or radiotherapy in a subject in need thereof comprising administering to the subject a therapeutically effective amount of an agent capable of depleting the population of CD163+ Tim4+ macrophages, wherein the cancer resistant is selected in the group consisting in ovarian cancer, breast cancer or pancreatic cancer.

In some embodiment, the cancer resistant is ovarian cancer.

In one embodiment, the cancer resistant is ovarian cancer resistant to immune checkpoint therapy.

As used herein, the term “resistance to immune checkpoint therapy, chemotherapy or radiotherapy” is used in its broadest context to refer to the reduced effectiveness of immune checkpoint therapy, chemotherapy or radiotherapy to inhibit the growth of a cell, kill a cell or inhibit one or more cellular functions, and to the ability of a cell to survive exposure to an agent designed to inhibit the growth of the cell, kill the cell or inhibit one or more cellular functions. The resistance displayed by a cell may be acquired, for example by prior exposure to the agent, or may be inherent or innate. The resistance displayed by a cell may be complete in that the agent is rendered completely ineffective against the cell, or may be partial in that the effectiveness of the agent is reduced. Accordingly, the term “resistant” refers to the repeated outbreak of cancer, or a progression of cancer independently of whether the disease was cured before said outbreak or progression.

A further object of the present invention relates to a method for preventing resistance to immune checkpoint therapy, chemotherapy or radiotherapy in a subject suffering from ovarian cancer, breast cancer or pancreatic cancer comprising administering to the subject a therapeutically effective amount of an agent capable of depleting the population of CD163+ Tim4+ macrophages.

In some embodiment, the invention relates to a method for preventing resistance to immune checkpoint therapy, radiotherapy or chemotherapy in a subject suffering from ovarian cancer comprising administering to the subject a therapeutically effective amount of an agent capable of depleting the population of CD163+ Tim4+ macrophages.

As used herein, the term “agent capable of depleting the population of CD163+ Tim4+ macrophages” refers to any compound that is able to deplete said populations. As used herein, the term “deplete” with respect to CD163+ Tim4+ macrophages, refers to a measurable decrease in the number of CD163+ Tim4+ macrophages in the subject's tumor. The reduction can be at least about 10%, e.g., at least about 20%, 30%, 40%, 50%, 60%, 70%, 80%, 90%, 95%, 96%, 97%, 98%, 99%, or more. In some embodiments, the term refers to a decrease in the number of CD163+ Tim4+ macrophages in a subject's tumor to an amount below detectable limits.

In some embodiments, the agent is an antibody having binding affinity for CD163 and that leads to the depletion of CD163+ Tim4+ macrophages in the subject's tumor. In particular, the antibody binds to the extracellular domain of CD163 as defined above.

In some embodiments, the agent is an antibody having binding affinity for Tim4 and that leads to the depletion of CD163+ Tim4+ macrophages in the subject's tumor. In particular, the antibody binds to the extracellular domain of Tim4 as defined above.

As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388.

As used herein the term “bind” indicates that the antibody has affinity for the surface molecule. The term “affinity”, as used herein, means the strength of the binding of an antibody to an epitope. The affinity of an antibody is given by the dissociation constant Kd, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

In some embodiments, the antibody is a humanized antibody. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference.

In some embodiments, the antibody is a fully human antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference.

In some embodiments, the antibody suitable for depletion of CD163+ Tim4+ macrophages mediates antibody-dependent cell-mediated cytotoxicity. As used herein the term “antibody-dependent cell-mediated cytotoxicity” or ‘ADCC” refer to a cell-mediated reaction in which non-specific cytotoxic cells (e.g., Natural Killer (NK) cells, neutrophils, and macrophages) recognize bound antibody on a target cell and subsequently cause lysis of the target cell. While not wishing to be limited to any particular mechanism of action, these cytotoxic cells that mediate ADCC generally express Fc receptors (FcRs).

As used herein “Fc region” includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, Va.). The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein. An Fc variant protein may be an antibody, Fc fusion, or any protein or protein domain that comprises an Fc region. Particularly preferred are proteins comprising variant Fc regions, which are non-naturally occurring variants of an Fc region. The amino acid sequence of a non-naturally occurring Fc region (also referred to herein as a “variant Fc region”) comprises a substitution, insertion and/or deletion of at least one amino acid residue compared to the wild type amino acid sequence. Any new amino acid residue appearing in the sequence of a variant Fc region as a result of an insertion or substitution may be referred to as a non-naturally occurring amino acid residue. Note: Polymorphisms have been observed at a number of Fc positions, including but not limited to Kabat 270, 272, 312, 315, 356, and 358, and thus slight differences between the presented sequence and sequences in the prior art may exist.

The terms “Fe receptor” or “FcR” are used to describe a receptor that binds to the Fc region of an antibody. The primary cells for mediating ADCC, NK cells, express FcγRIII, whereas monocytes express FcγRI, FcγRII, FcγRIII and/or FcγRIV. FcR expression on hematopoietic cells is summarized in Ravetch and Kinet, Annu. Rev. Immunol., 9:457-92 (1991). To assess ADCC activity of a molecule, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecules of interest may be assessed in vivo, e.g., in an animal model such as that disclosed in Clynes et al., Proc. Natl. Acad. Sci. (USA), 95:652-656 (1998). As used herein, the term Effector cells” are leukocytes which express one or more FcRs and perform effector functions. The cells express at least FcγRI, FCγRII, FcγRIII and/or FcγRIV and carry out ADCC effector function. Examples of human leukocytes which mediate ADCC include peripheral blood mononuclear cells (PBMC), natural killer (NK) cells, monocytes, cytotoxic T cells and neutrophils.

In some embodiments, the antibody suitable for depletion of CD163+ Tim4+ macrophages is a full-length antibody. In some embodiments, the full-length antibody is an IgG1 antibody. In some embodiments, the full-length antibody is an IgG3 antibody.

In some embodiments, the antibody suitable for depletion of CD163+ Tim4+ macrophages comprises a variant Fc region that has an increased affinity for FcγRIA, FcγRIIA, FcγRIIB, FcγRIIIA, FcγRIIIB, and FcγRIV. In some embodiments, the antibody of the present invention comprises a variant Fc region comprising at least one amino acid substitution, insertion or deletion wherein said at least one amino acid residue substitution, insertion or deletion results in an increased affinity for FcγRIA, FcγRIIA, FcγRIIB, FcγRIIIA, FcγRIIIB, and FcγRIV, In some embodiments, the antibody of the present invention comprises a variant Fc region comprising at least one amino acid substitution, insertion or deletion wherein said at least one amino acid residue is selected from the group consisting of: residue 239, 330, and 332, wherein amino acid residues are numbered following the EU index. In some embodiments, the antibody of the present invention comprises a variant Fc region comprising at least one amino acid substitution wherein said at least one amino acid substitution is selected from the group consisting of: S239D, A330L, A330Y, and 1332E, wherein amino acid residues are numbered following the EU index.

In some embodiments, the glycosylation of the antibody suitable for depletion of CD163+ Tim4+ macrophages is modified. For example, an aglycosylated antibody can be made (i.e., the antibody lacks glycosylation). Glycosylation can be altered to, for example, increase the affinity of the antibody for the antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al. Additionally or alternatively, an antibody can be made that has an altered type of glycosylation, such as a hypofucosylated or non-fucosylated antibody having reduced amounts of or no fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the present invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation or are devoid of fucosyl residues. Therefore, in some embodiments, the human monoclonal antibodies of the present invention may be produced by recombinant expression in a cell line which exhibit hypofucosylation or non-fucosylation pattern, for example, a mammalian cell line with deficient expression of the FUT8 gene encoding fucosyltransferase. PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al, 2002 J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al, 1999 Nat. Biotech. 17: 176-180). Eureka Therapeutics further describes genetically engineered CHO mammalian cells capable of producing antibodies with altered mammalian glycosylation pattern devoid of fucosyl residues (http://www.eurekainc.com/a&boutus/companyoverview.html). Alternatively, the human monoclonal antibodies of the present invention can be produced in yeasts or filamentous fungi engineered for mammalian-like glycosylation pattern and capable of producing antibodies lacking fucose as glycosylation pattern (see for example EP1297172B 1

In some embodiments, the antibody suitable for depletion of CD163+ Tim4+ macrophages mediates complement dependant cytotoxicity. “Complement dependent cytotoxicity” or “CDC” refers to the ability of a molecule to initiate complement activation and lyse a target in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santaro et al., J. Immunol. Methods, 202:163 (1996), may be performed.

In some embodiments, the antibody suitable for depletion of CD163+ Tim4+ macrophages mediates antibody-dependent phagocytosis. As used herein, the term “antibody-dependent phagocytosis” or “opsonisation” refers to the cell-mediated reaction wherein nonspecific cytotoxic cells that express FcγRs recognize bound antibody on a target cell and subsequently cause phagocytosis of the target cell.

In some embodiments, the antibody suitable for depletion of CD163+ Tim4+ macrophages is a multispecific antibody comprising a first antigen binding site directed against CD163 and at least one second antigen binding site directed against an effector cell as above described. In some embodiments, the antibody suitable for depletion of CD163+ Tim4+ macrophages is a multispecific antibody comprising a first antigen binding site directed against Tim4 and at least one second antigen binding site directed against an effector cell as above described. In said embodiments, the second antigen-binding site is used for recruiting a killing mechanism such as, for example, by binding an antigen on a human effector cell. In some embodiments, an effector cell is capable of inducing ADCC, such as a natural killer cell. For example, monocytes, macrophages, which express FcRs, are involved in specific killing of target cells and presenting antigens to other components of the immune system. In some embodiments, an effector cell may phagocytose a target antigen or target cell. The expression of a particular FcR on an effector cell may be regulated by humoral factors such as cytokines. An effector cell can phagocytose a target antigen or phagocytose or lyse a target cell. Suitable cytotoxic agents and second therapeutic agents are exemplified below, and include toxins (such as radiolabeled peptides), chemotherapeutic agents and prodrugs. In some embodiments, the second binding site binds to a Fc receptor as above defined. In some embodiments, the second binding site binds to a surface molecule of NK cells so that said cells can be activated. In some embodiments, the second binding site binds to NKp46. Exemplary formats for the multispecific antibody molecules of the present invention include, but are not limited to (i) two antibodies cross-linked by chemical heteroconjugation, one with a specificity to a specific surface molecule of ILC and another with a specificity to a second antigen; (ii) a single antibody that comprises two different antigen-binding regions; (iii) a single-chain antibody that comprises two different antigen-binding regions, e.g., two scFvs linked in tandem by an extra peptide linker; (iv) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (v) a chemically-linked bispecific (Fab′)2 fragment; (vi) a Tandab, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain” in Protein Kinase A, which, when applied to Fabs, can yield a trivaient bispecific binding protein consisting of two identical Fab fragments linked to a different Fab fragment; (ix) a so-called Scorpion molecule, comprising, e.g., two scFvs fused to both termini of a human Fab-arm; and (x) a diabody. Another exemplary format for bispecific antibodies is IgG-like molecules with complementary CH3 domains to force heterodimerization. Such molecules can be prepared using known technologies, such as, e.g., those known as Triomab/Quadroma (Trion Pharma/Fresenius Biotech), Knob-into-Hole (Genentech), CrossMAb (Roche) and electrostatically-matched (Amgen), LUZ-Y (Genentech), Strand Exchange Engineered Domain body (SEEDbody)(EMD Serono), Biclonic (Merus) and DuoBody (Genmab A/S) technologies.

In some embodiments, the antibody suitable for depletion of CD163+ Tim4+ macrophages is a multispecific antibody comprising a first antigen binding site directed against CD163, and at least one second antigen binding site directed against Tim4.

In some embodiments, the antibody suitable for depletion of CD163+ Tim4+ macrophages is a bispecific antibody comprising a first antigen binding site directed against CD163, and a second antigen binding site directed against Tim4.

In some embodiments, the antibody suitable for depletion of CD163+ Tim4+ macrophages is a multispecific antibody comprising a first antigen binding site directed against CD163, a second antigen binding site directed against Tim4 and at least one third antigen binding site directed against an effector cell as above described.

In some embodiments, the antibody suitable for depletion of CD163+ Tim4+ macrophages is conjugated to a therapeutic moiety, i.e. a drug. The therapeutic moiety can be, e.g., a cytotoxin, a chemotherapeutic agent, a cytokine, an immunosuppressant, an immune stimulator, a lytic peptide, or a radioisotope. Such conjugates are referred to herein as an “antibody-drug conjugates” or “ADCs”.

In some embodiments, the antibody suitable for depletion of CD163+ Tim4+ macrophages is conjugated to a cytotoxic moiety. The cytotoxic moiety may, for example, be selected from the group consisting of taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; a tubulin-inhibitor such as maytansine or an analog or derivative thereof; an antimitotic agent such as monomethyl auristatin E or F or an analog or derivative thereof; dolastatin 10 or 15 or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof an antimetabolite such as methotrexate, 6 mercaptopurine, 6 thioguanine, cytarabine, fludarabin, 5 fluorouracil, decarbazine, hydroxyurea, asparaginase, gemcitabine, or cladribine; an alkylating agent such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C; a platinum derivative such as cisplatin or carboplatin; duocarmycin A, duocarmycin SA, rachelmycin (CC-1065), or an analog or derivative thereof; an antibiotic such as dactinomycin, bleomycin, daunorubicin, doxorubicin, idarubicin, mithramycin, mitomycin, mitoxantrone, plicamycin, anthramycin (AMC)); pyrrolo[2,1-c][1,4]-benzodiazepines (PDB); diphtheria toxin and related molecules such as diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins such as PAPI, PAPII, and PAP-S, Momordica charantia inhibitor, curcin, crotin, Sapaonaria officinalis inhibitor, gelonin, mitogellin, restrictocin, phenomycin, and enomycin toxins; ribonuclease (RNase); DNase I, Staphylococcal enterotoxin A; pokeweed antiviral protein; diphtherin toxin; and Pseudomonas endotoxin.

In some embodiments, the antibody suitable for depletion of CD163+ Tim4+ macrophages is conjugated to an auristatin or a peptide analog, derivative or prodrug thereof. Auristatins have been shown to interfere with microtubule dynamics, GTP hydrolysis and nuclear and cellular division (Woyke et al (2001) Antimicrob. Agents and Chemother. 45(12): 3580-3584) and have anti-cancer (U.S. Pat. No. 5,663,149) and antifungal activity (Pettit et al., (1998) Antimicrob. Agents and Chemother. 42: 2961-2965. For example, auristatin E can be reacted with para-acetyl benzoic acid or benzoylvaleric acid to produce AEB and AEVB, respectively. Other typical auristatin derivatives include AFP, MMAF (monomethyl auristatin F), and MMAE (monomethyl auristatin E). Suitable auristatins and auristatin analogs, derivatives and prodrugs, as well as suitable linkers for conjugation of auristatins to Abs, are described in, e.g., U.S. Pat. Nos. 5,635,483, 5,780,588 and 6,214,345 and in International patent application publications WO02088172, WO2004010957, WO2005081711, WO2005084390, WO2006132670, WO03026577, WO200700860, WO207011968 and WO205082023.

In some embodiments, the antibody suitable for depletion of CD163+ Tim4+ macrophages is conjugated to pyrrolo[2,1-c][1,4]-benzodiazepine (PDB) or an analog, derivative or prodrug thereof. Suitable PDBs and PDB derivatives, and related technologies are described in, e.g., Hartley J. A. et al., Cancer Res 2010; 70(17): 6849-6858; Antonow D. et al., Cancer J 2008; 14(3): 154-169; Howard P. W. et al., Bioorg Med Chem Lett 2009; 19: 6463-6466 and Sagnou et al., Bioorg Med Chem Lett 2000; 10(18): 2083-2086. In some embodiments, the antibody is conjugated to pyrrolobenzodiazepine (PBD) as typically described in WO2017059289.

In some embodiments, the antibody suitable for depletion of CD163+ Tim4+ macrophages is conjugated to a cytotoxic moiety selected from the group consisting of an anthracycline, maytansine, calicheamicin, duocarmycin, rachelmycin (CC-1065), dolastatin 10, dolastatin 15, irinotecan, monomethyl auristatin E, monomethyl auristatin F, a PDB, or an analog, derivative, or prodrug of any thereof.

In some embodiment, the antibody suitable for depletion of CD163+ Tim4+ macrophages is conjugated to duocarmycin.

In some embodiment, the antibody suitable for depletion of CD163+ Tim4+ macrophages is an anti-TIM4 antibody conjugated to duocarmycin.

In some embodiment, the antibody suitable for depletion of CD163+ Tim4+ macrophages is an anti-TIM4 IgG conjugated to duocarmycin.

In some embodiments, the antibody suitable for depletion of CD163+ Tim4+ macrophages is conjugated to an anthracycline or an analog, derivative or prodrug thereof. In some embodiments, the antibody is conjugated to maytansine or an analog, derivative or prodrug thereof. In some embodiments, the antibody is conjugated to calicheamicin or an analog, derivative or prodrug thereof. In some embodiments, the antibody is conjugated to duocarmycin or an analog, derivative or prodrug thereof. In some embodiments, the antibody is conjugated to rachelmycin (CC-1065) or an analog, derivative or prodrug thereof. In some embodiments, the antibody is conjugated to dolastatin 10 or an analog, derivative or prodrug thereof. In some embodiments, the antibody is conjugated to dolastatin 15 or an analog, derivative or prodrug thereof. In some embodiments, the antibody is conjugated to monomethyl auristatin E or an analog, derivative or prodrug thereof. In some embodiments, the antibody is conjugated to monomethyl auristatin F or an analog, derivative or prodrug thereof. In some embodiments, the antibody is conjugated to pyrrolo[2,1-c][1,4]-benzodiazepine or an analog, derivative or prodrug thereof. In some embodiments, the antibody is conjugated to irinotecan or an analog, derivative or prodrug thereof.

In some embodiments, the antibody suitable for depletion of CD163+ Tim4+ macrophages is conjugated to a nucleic acid or nucleic acid-associated molecule. In one such embodiment, the conjugated nucleic acid is a cytotoxic ribonuclease (RNase) or deoxy-ribonuclease (e.g., DNase I), an antisense nucleic acid, an inhibitory RNA molecule (e.g., a siRNA molecule) or an immunostimulatory nucleic acid (e.g., an immunostimulatory CpG motif-containing DNA molecule). In some embodiments, the antibody is conjugated to an aptamer or a ribozyme.

Techniques for conjugating molecule to antibodies, are well-known in the art (See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies ‘84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.) Typically, the nucleic acid molecule is covalently attached to lysines or cysteines on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106.; Junutula, J. R., Flagella, K. M., Graham, R. A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target humanepidermal growth factor receptor 2-positive breast cancer. Clin. Cancer Res. 16, 4769-4778.). Junutula et al. (2008) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established (Axup et al., 2012). In particular the one skilled in the art can also envisage Fc-containing polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase, can covalently crosslink with an amine donor agent (e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (WO 2012059882).

In some embodiment, the agent capable of depleting the population of CD163+ Tim4+ macrophages can be encapsulating in long circulating liposomes, and more particular in lipid nanoparticles (LNP).

As used herein, the term “lipid nanoparticle” has its general meaning in the art and refers to a non-viral gene delivery system. The size of LNPs is one of the essential factors affecting drug delivery efficiency and therapeutic efficiency. The lipid nanoparticles can be designed to improve the pharmacokinetics and biodistribution of the agent.

Typically, the agent capable of depleting the population of CD163+ Tim4+ macrophages is administered to the patient in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

### Composition for Use

An aspect of the invention relates to a composition comprising an antibody having binding affinity for CD163, for use in the treatment of ovarian and/or pancreatic cancer.

In a preferred embodiment, the antibody is an antibody-drug conjugate.

In an embodiment, the anti-CD163 antibody leads to the depletion of CD163+/Tim4+ macrophages in the subject's tumor. As previously mentioned the anti-CD163 antibody may deplete cell populations which are CD163⁺/Tim4⁻ and cell populations which are CD163⁺/Tim4⁺.

In an embodiment, the cancer is ovarian cancer.

In another embodiment, cancer is pancreatic cancer.

In an embodiment, the antibody binds to the extracellular domain of CD163.

In yet an embodiment, the composition further comprises an antibody having binding affinity for Tim4. In a related embodiment, the Tim4 antibody leads to the depletion of CD163+/Tim4+ macrophages in the subject's tumor. As previously mentioned the anti-Tim4 antibody may deplete cell populations which are CD163⁺/Tim4⁺ and cell populations which are CD163⁻/Tim4⁺. When combined depletion of all CD163⁺ cell populations and Tim4⁺ cell populations will take place. As also previously outlined, these different cell populations are considered important to target alone or in combination, preferably in combination.

Examples 11 and 12 shows in in vitro and in vivo cytotoxicity data with an anti-Tim4 antibody conjugated to duocarmycin.

In a preferred embodiment, the Tim4 antibody is an antibody-drug conjugate.

In a further embodiment, the Tim4 antibody binds to the extracellular domain of Tim4.

In an embodiment, the antibodies (the CD163 antibody and/or the Tim4 antibody) mediate antibody-dependent cell-mediated cytotoxicity.

In yet an embodiment, the antibody is a multispecific antibody comprising a first antigen binding site directed against CD163.

In another embodiment, the antibody (The CD163 and/or Tim4) is an antibody-drug conjugate. In a related embodiment, the antibody is conjugated to a cytotoxic moiety. In yet a related embodiment, the antibody is conjugated to doxorubicin and/or duocarmycin.

Another aspect of the invention relates to a composition comprising an anti-Tim4 antibody for use as a medicament. Examples 11 and 12 show in in vitro and in vivo cytotoxicity data with an anti-Tim4 antibody conjugated to duocarmycin. As previously mentioned the anti-Tim4 antibody may deplete cell populations which are CD163⁺/Tim4⁺ and cell populations which are CD163⁻/Tim4⁺.

In an embodiment, the composition is for use in the treatment of cancer, such as ovarian and/or pancreatic cancer. In a preferred embodiment, the cancer is pancreatic cancer. Example 12 shows in vivo data for a pancreatic cancer mice model using a Tim4 antibody.

In another preferred embodiment, the anti-Tim4 antibody is conjugated to a cytotoxic moiety. In yet a related embodiment, the antibody is conjugated to doxorubicin and/or duocarmycin.

### Combination

Another aspect of the invention relates to a combination comprising an antibody having binding affinity for CD163 and an antibody having binding affinity for Tim4. As previously mentioned the anti-Tim4 antibody may deplete cell populations which are CD163⁺/Tim4⁺ and cell populations which are CD163⁻/Tim4⁺. The anti-CD163 antibody may deplete cell populations which are CD163⁺/Tim4⁻ and cell populations which are CD163⁺/Tim4⁺.

When combined depletion of all CD163⁺ cell populations and Tim4⁺ cell populations will take place. As also previously outlined, these different cell populations are considered important to target alone or in combination, preferably in combination.

In an embodiment, the antibodies are antibody-drug conjugates. Preferably, the CD163 antibody and/or the Tim4 antibody are antibody-drug conjugates, more preferably both.

In an aspect, the combination is for use as a medicament.

In yet an aspect the combination is for use in the treatment of ovarian and/or pancreatic cancer.

### Kit of Parts

An aspect of the invention relates to a kit of parts comprising
a first container comprising an antibody having binding affinity for CD163;
a second container comprising an antibody having binding affinity for Tim4.

As previously mentioned, the anti-Tim4 antibody may deplete cell populations which are CD163⁺/Tim4⁺ and cell populations which are CD163⁻/Tim4⁺. The anti-CD163 antibody may deplete cell populations which are CD163⁺/Tim4⁻ and cell populations which are CD163⁺/Tim4⁺.

When combined depletion of all CD163⁺ cell populations and Tim4⁺ cell populations may take place. As also previously outlined, these different cell populations are considered important to target alone or in combination, preferably in combination.

In an embodiment, the antibodies are antibody-drug conjugates. Preferably, the CD163 antibody and/or the Tim4 antibody are antibody-drug conjugates, more preferably both.

In an aspect, the kit of parts is for use as a medicament.

In yet an aspect the kit of parts is for use in the treatment of ovarian and/or pancreatic cancer.

### CLAUSES

The present invention may also be defined by the following clauses.
1. A method of treating a cancer selected in the group consisting of ovarian cancer, breast cancer and pancreatic cancer in a subject in need thereof comprising administering to the subject a therapeutically effective of an agent capable of depleting the population of CD163+ Tim4+ macrophages.
2. The method of clause 1, wherein the cancer is ovarian cancer.
3. The method of clause 2, wherein the cancer is ovarian cancer resistant to immune checkpoint therapy, radiotherapy or chemotherapy
4. The method of any of clause 1 to 3, wherein the agent capable of depleting the population of CD163+ Tim4+ macrophages of the invention is administered in combination with an agent capable of depleting the population of TAM that only express CD163.
5. A method of preventing resistance to immune checkpoint therapy, radiotherapy or chemotherapy in a subject suffering from a cancer selected in the group consisting of ovarian cancer, breast cancer or pancreatic cancer comprising administering to the subject a therapeutically effective amount of an agent capable of depleting the population of CD163+ Tim4+ macrophages.
6. The method according to any preceding clauses wherein the agent is an antibody having binding affinity for CD163 and that leads to the depletion of CD163+ Tim4+ macrophages in the subject's tumor.
7. The method of claim 6, wherein the antibody binds to the extracellular domain of CD163.
8. The method according to any preceding clauses, wherein the agent is an antibody having binding affinity for Tim4 and that leads to the depletion of CD163+ Tim4+ macrophages in the subject's tumor.
9. The method of clause 8, wherein the antibody binds to the extracellular domain of Tim4.
10. The method of clause 6 or 8 wherein the antibody mediates antibody-dependent cell-mediated cytotoxicity.
11. The method of clause 6 wherein the antibody is a multispecific antibody comprising a first antigen binding site directed against CD163.
12. The method of clause 6 or 8, wherein the antibody is a multispecific antibody comprising a first antigen binding site directed against CD163 and a second antigen binding site directed against Tim4.
13. The method of clause 6 or 8, wherein the antibody is an antibody-drug conjugate.
14. The method of clause 13, wherein the antibody is conjugated to a cytotoxic moiety.
15. The antibody of claim 14, wherein the antibody is conjugated to duocarmycin.

The invention will be further illustrated by the following figures and examples. However, these examples and figures should not be interpreted in any way as limiting the scope of the present invention.

### FIGURES

FIG. 1: Embryonic origin of tissue-resident CD163⁺ Time macrophages in omentum. A. Chimerism was calculated as proportion of CD45.1/.2+CD169hi Lyve-1+ macrophages relative to CD45.1/.2+ expression among Ly6Chi blood monocytes. Data is represented as mean+/−SEM of n=5; ***p<0.001. B. Mice were injected with ID8-luc i.p and after 8 weeks 1 mg tamoxifen was administered by oral gavage and RFP expression was analyzed in CD169^{hi} Lyve-1⁺ macrophages subsets (P1: CD163⁺ Tim4⁺; P2: CD163⁺ Tim4⁻; P3: CD163⁻ Tim4⁻; P4: CD163⁻ Tim4⁺) 10 days later. Data is represented as mean+/−SEM of n=4. C. Omentum was harvested and analyzed by flow cytometry 8 weeks after birth and % YFP⁺ cells was calculated relative to YFP⁺ microglia. Data is represented as mean+/−SEM of n=6.

FIG. 2: Specific depletion of CD163⁺ Time tissue-resident macrophages prevents metastatic spread of ovarian cancer. A. Flow cytometry analysis of CD163hi Lyve-1+ macrophages in omentum of Cd163-Csflr^{DTR} and Csflr^{LSL-DTR} mice 10 weeks after injection of ID8 cells. B. Omentum weight, C. total tumor cells in ascites and D. ascites volume in Cd163-Csflr^{DTR} and CsflrL^{SL-DTR} mice treated with DT. E. Ex vivo bioluminescence analysis of metastases on the diaphragm of Cd163-Csflr^{DTR} and CsflrL^{SL-DTR}. Data is represented as mean+/−SEM of n=7; *p<0.05, **p<0.01 and ***p<0.001. F. Tumor burden monitored by in vivo bioluminescent imaging. G. Flow cytometry analysis of P1-P4 macrophages in omentum after thereapeutic depletion of CD163+ cells. H. Omentum weight, I. total tumor cells in ascites and J. ascites volume. Data is represented as mean+/−SEM of n=6; *p<0.05, **p<0.01 and ***p<0.001.

FIG. 3: Ovarian cancer cells in ascites acquire cancer stem cell (CSC) characteristics. A. Analysis of ALDH activity in ID8 and ID8-A11 cells by flow cytometry using the ALDEFLUOR assay. ALDH+ cells were gated using DEAB treated cells as negative control, relative ALDH activity was calculated by measuring the median fluorescence intensity of aldefluor. Data is represented as mean+/−SEM of n=5 (ID8) or n=8 (ID8-A11); *p<0.05 and **p<0.01. B. Analysis of tumorigenic potential of ID8 and ID8-A11 cells in vivo; total tumor burden was monitored by in vivo bioluminescence imaging. Ex vivo analysis of tumor burden in C. omentum and D. ascites at 30 days after transplantation of ID8-luc or ID8-A11 cells. Data is represented as mean+/−SEM of n=6 (ID8) or n=5 (ID8-A11); *p<0.05, **p<0.01, ***p<0.001 and ****p<0.0001.

FIG. 4: CD163⁺ Tim4⁺ tissue-resident macrophages promote the CSC-like phenotype of ovarian cancer cells. A. Flow cytometry analysis of CD163^{hi} Lyve-1⁺ macrophages (P1: CD163⁺ Tim4⁺; P2: CD163⁺ Tim4⁻; P3: CD163⁻ Tim4⁻; P4: CD163⁻ Tim4⁺) in omentum at 10 weeks after prophylactic treatment with αCD163-dxr. B. Omentum weight, C. total tumor cells in ascites and D. ascites volume at 10 weeks after prophylactic treatment with αCD163-dxr. E. Ex vivo bioluminescence analysis of metastases on the diaphragm. Data is represented as mean+/−SEM of n=8; *p<0.05 and **p<0.01. F. Flow cytometry analysis identifying the proportion of malignant cells expressing specific CSC markers; CD54, CD55, CD106 and CD117. G. Gene expression analysis of Gata3, Stat3, Wnt5a and Mertk in tumor cells from omentum after specific depletion of CD163⁺ Tim4⁺ (P1) macrophages. Data is represented as mean+/−SEM of n=6; *p<0.05, **p<0.01, ***p<0.001 and ****p<0.0001.

FIG. 5: (A) Effect of CD163+ TAM depletion in the murine pancreatic ductal adenocarcinoma (PDAC) model P48-Cre; LSL-KrasG12D; LSL-Trp53R172H. From 6 weeks of age mice were treated with CD163-targeted cytotoxic LNP (αCD163-dxr; 1 mg/kg dxr), non-targeted LNP (ctrllgG-dxr), empty targeted-LNP (αCD163-LNP) or PBS alone (all n=8) i.v. twice a week. (B) Relative gene expression (2-ACT) of Cd163 in snap frozen tumor tissue harvested at endpoint (20 weeks).

FIG. 6: Duocarmycin conjugated anti-mouse Tim4 or control IgG was added to the Tim4 expressing cells in vitro.

FIG. 7: Frequency of Tim4 expressing large peritoneal macrophages (LPM) (A), and small peritoneal macrophages (SPM) (B), 24 hrs after intra peritoneal injection of Tim4 antibody conjugated to duocarmycin.

### EXAMPLES

### Example 1—Material & Methods

Mouse Breeding and Ovarian Cancer Model

Tg(Csflr-LSL-HBEGFmCherry) (Schreiber et al., 2013), Rosa26^{LSL-YFP}, Cx3Crl^{CreER} and Cx3Clr^{GFP/+} were obtained from the Jackson Laboratory (Bar Harbor, Me., US). C57BL/6J mice were obtained from Janvier Labs (Saint-Berthevin, FR). Ccr2^{-/-} mice and Rosa26^{LS-tdRFP} mice were gifts from Bernard Malissen (Centre d'Immunologie Marseille Luminy, Marseille, France). Cd163^{iCre} mice were generated from modified ES cells on a C57BL/6 background as described (Etzerodt et al, in press). In brief; a FlpO-NeoR cassette encoding IRES-iCre was inserted in the 3′UTR of the CD163 gene using homologous recombination and used to generate chimeric mice that were subsequently crossed to Flp deleter mice to facilitate removal of NeoR cassette. To mimick peritoneal spread of epithelial ovarian cancer, 1×10⁶ ID8-Luc cells were injected i.p in 500 μl sterile PBS pH 7.4. Tumor burden was estimated weekly by injecting mice i.p. with 100 mg/kg d-Luciferin followed by in vivo bioluminescence imaging using an IVIS Spectrum imager (PerkinElmer). For visualizing infiltration of ID8-cells in omentum, ID8 EOC cells were prelabeled with Qtracker 705 cell labeling kit (Thermo Fisher) prior to i.p. injections in accordance with manufactures instructions. For therapeutic treatment with lipid nanoparticles (LNP) mice were injected with 100 μl of LNP (1 mg/kg dxr) by retroorbital injection starting from 5 weeks after i.p injection of 1×10⁶. For prophylactic treatment with LNP or diphteria toxin, mice were injected i.p with either 200 μl LNP (1 mg/kg dxr) or 200 μl diptheria toxin (4 ng/kg) starting from 6 days prior to inoculation of tumor cells. All mice were euthanized at the indicated times and peritoneal lavages and tissues were collected for cytometric analysis or imaging analysis. Briefly, 3 ml of ice-cold PBS with 2 mM EDTA, pH 8.0 was injected intraperitoneally and after a careful massage to detach all the cells in the cavity, peritoneal fluid was collected through a 23G syringe. Tubes were weighed to determine the recovered lavage volume and the cell density was assessed using a hemocytometer. All mice were housed at the animal facility at Centre d'immunologie Marseille-Luminy with water and food ad libitum and 12h night/daylight cycle. All animal experiments were approved and carried out in accordance with the limiting principles for using animal in testing (the three R's, replacement, reduction and refinement) and approved by the French Ministry of Higher Education and Research.

Fate-Mapping Experiments

Genetic fate-mapping using Cx3Crl^{CreER}:R26-YFP mice was performed as previously described (Mossadegh-Keller et al., 2017); pregnant females were pulse-labeled at E16.5 by intraperitoneal injection of 0.1 mg/kg tamoxifen and 0.05 mg/kg progesterone. For fate-mapping in adult tumor bearing mice, Cx3Crl^{CreER}:R26-tdRFP mice were injected with 1×10⁶ ID8-luc cells i.p. and after 6 weeks pulse-labeled with a single dose of 2 mg tamoxifen dissolved in 200 μl corn oil by oral gavage. Generation of shielded chimeras was performed as previously described (Goossens et al., 2019); CD45.1 congenic mice were anaesthetized with Ketamine (150 mg/kg) and Xylazine (10 mg/kg) and placed in 6 mm thick lead cylinders, exposing only the hind legs. With the abdominal area protected, mice were irradiated with 9 Gy and reconstituted with 10⁷ bone marrow cells from CD45.1/.2 mice. After 5 weeks, chimerism of blood leukocytes was assessed by flow cytometry.

Flow Cytometry and Cell Sorting

Single cell suspensions were prepared from omentum by digesting the tissue in RPMI 1640 medium with 1 mg/ml Collagenase II (Sigma Aldrich), 50 μg/ml DNAseI (Roche, Hvidovre, DK) and 0.1% (w/v) BSA for 30 min at 37° C. with gentle agitation. Cell suspensions were subsequently passed through 70 μm cell strainer (BD Biosciences, FR) and collected by centrifugation. Blood and ascitic cells harvested by peritoneal lavage were used without further processing. For red blood cell (RBC) lysis, cell suspensions were incubated with 0.85% NH₄Cl for 2 min at RT, collected by centrifugation and resuspended directly in FACS buffer (1×PBS pH 7.4, 1 mM EDTA pH 8.0, 3% FCS and 0.1% NaN₃). For flow cytometry, single-cell suspensions were incubated at 4° C. for 10 min with 2.4G2 antibody for Fc receptor blocking followed by incubation with the specified antibodies (see Table 1 for details) for 30 min at 4° C. Prior to analysis, cells were incubated with Sytox Blue (Thermo Fischer Scientific, FR) to discriminate dead cells and filtered through a 70 μm cell strainer. ALDH activity in tumor cells was measured using the ALDEFLUOR Kit (Stem cell Technologies), in accordance with manufactures instructions. In brief, 2×10⁶ cells were incubated with 5 μl ALDEFLUOR stock solution or 5 μl ALDEFLUOR stock solution in combination with 5 μl of the ALDH inhibitor DEAB and incubated for 30 min at 37° C. ALDH activity was subsequently measured by flow cytometry relative to DEAB treated control cells. All flow cytometry analysis was performed with either BD FACS LSR-2 or Fortessa X-20 flow cytometers whereas cell sorting was performed with BD FACS Aria SORP. All cytometers were equipped with a 350 nm laser (BD Biosciences). Subsequent data analysis was performed using FlowJo software V10.4 for Mac (Tree Star).

Liposome Preparation

Long-circulating liposomes encapsulating doxorubicin were prepared and modified for CD163 targeting as previously described (Etzerodt et al., 2012)(Fritze et al., 2006). Briefly, liposome formulations were formed using the ethanol-injection method from a mixture of HSPC, mPEG2000-PE and Cholesterol (molar ratio of 55:40:5) (Lipoid GmBH, Ludwigshafen, Germany and Sigma Aldrich). Lipids were dissolved in EtOH at 65° C. for 15 min followed by hydration (to 10% EtOH) for 1 h at 65° C. in aqueous buffer suitable for further downstream applications. Liposomes were sized by extrusion 25 times through a 0.1 μm filter using the Avanti mini-extruder kit (Avanti Polar Lipids, AL, US) and dialyzed twice against 150 mM NaCl (0.9% NaCl) with second dialysis being over night at 4° C. For remote loading of doxorubicin, lipid was hydrated in 300 mM (NH₄)₁HPO₃. Following extrusion and dialysis, (NH₄)₁HPO₃ containing liposomes were mixed with doxorubicin-HCl for 30 min at 65° C. at a doxorubicin:lipid ratio at 1:5. Lipid content, drug content and encapsulation efficiency was subsequently estimated from high-pressure size-exclusion chromatography (UV absorbance 210 nm) using a Dionex Ultimate3000 HPLC system (Thermo Fischer Scientific, Hvidovre, Denmark) equipped with Ascentis C18 column (Sigma Aldrich). Liposome size was estimated using dynamic light scattering and the DynaPro NanoStar system (Wyatt Technology Europe GmbH, Dernbach, Germany). Modification of liposomes for CD163 targeting was done as described previously using the post-insertion method of αCD163 antibody, clone 3E10B10 (Etzerodt et al., 2012; Torchilin et al., 2001).

Immunohistochemistry and Whole-Mount Immunofluorescence Imaging

Omentum was fixed in 1% formalin and either embedded in OCT for cryostat sectioning or used directly for whole mount imaging. For histological analysis, 10 μm cryostat sections were mounted on glass slides and stained with hematoxylin and eosin and visualized on an upright microscope equipped with a 10× objective. For whole mount imaging, omental tissue was incubated with with anti-CD163-ATT0565 (Etzerodt et al., 2013), anti-CD169-eFluor660 (Clone Ser4; eBioscience), anti-CD45.2-Alexa488 (Clone 104; Biolegend) and Tim4-Alexa647 (RMT54-4; Biolegend) in 0.1M Tris pH 7.2, 1% Triton X-100, 0.5% BSA overnight at 4° C. Nuclei were visualized with Hoechst 33342 (Sigma Aldrich). Tissue was subsequently mounted in RapiClear 1.47 for tissue clearing on glass slides with 0.2 mm iSpacer (SunJin Lab Co. Hsinchu City, Taiwan). Images were acquired on a Zeiss LSM780 confocal microscope (Carl Zeiss Microscopy GmbH, Jena, DE) using spectral unmixing with a 10× or 20× objective.

Spheroid Formation Assay

Tumor cells in ascites were enriched by depleting leukocytes in peritoneal lavage using the CD45.2 magnisort kit (eBioscience) and seeded at 40,000 cells per well in ultra-low attachment 96 well plates (Corning Life Science, UK) in DMEM supplemented with 4% heat-inactivated FCS. Formation of spheroids was subsequently monitored by microscopy using an inverted microscope equipped with a 4× objective.

RNA Sequencing and Bioinformatics Analysis

Library preparation and RNA sequencing (RNAseq) was performed by the GenomEast platform at Institut de Génétique et Biologie Moléculaire et Cellulaire, Strasbourg, France. Full length cDNA was generated using Clontech SMART-Seq v4 Ultra Low Input RNA kit (Takara Bio Europe, Saint Germain en Laye, France) according to manufacturer's instructions from 500 cells isolated by cell sorting in PBS buffer containing RNAses inhibitor. cDNA was amplified with 14 cycles of PCR for cDNA amplification by Seq-Amp polymerase. Six hundred pg of pre-amplified cDNA were then used as input for Tn5 transposon tagmentation by the Nextera XT DNA Library Preparation Kit (96 samples) (Illumina, San Diego, Calif.) followed by 12 cycles of library amplification. Following purification with Agencourt AMPure XP beads (Beckman-Coulter, Villepinte, France), the size and concentration of library DNA were assessed on an Agilent 2100 Bioanalyzer. Libraries were sequenced on an Illumina HiSeq4000 platform generating 50 bp reads. Samples were trimmed to remove TruSeq adapters using BBduk (Bushnell, 2014) and subsequently mapped to the mouse genome assembly version mm10 using STAR version 2.5.3a (Dobin et al., 2012) with junction annotation from Ensembl version 79 (Yates et al., 2016). Gene counts obtained directly from STAR were used in gene expression analysis with DESeq2 (Love et al., 2014) using cqn (Hansen et al., 2012) derived normalization factors. Self-organizing maps (SOM) clustering analysis was performed using the “kohonen” R pacakage (Wehrens and Buydens, 2007; Wehrens and Kruisselbrink, 2018), while comparative gene ontology analysis was carried out using clusterProfiler (Yu et al., 2012). Heatmaps and hierarchical clustering was generated using the One minus Pearson correlation and PCA plots with network analysis (using Pearson correlation) to show the 3 nearest neighbors were generated using Qlucore Omics Explorer (Qlucore AB, Lund, SE).

Gene Expression Analysis

Total RNA was purified from sorted cell populations using the RNeasy Micro Kit (Qiagen, Hilden, DE) and concentration determined using the Quant-IT RiboGreen RNA assay kit (Thermo Fischer Scientific). First strand cDNA synthesis was performed with High Capacity cDNA Reverse Transcriptase Kit (Thermo Fischer Scientific) followed by preamplification of genes of interest using the Fluidigm PreAmp Master Mix (Fluidigm Europe B.V. Amsterdam, NL) with 25 ng of total RNA and in accordance with the manufacturer's instructions. Exon-spanning primers to amply genes of interest were designed using Primer-Blast (see Table 2 for details). To increase sensitivity, genes of interest were pre-amplified by 12 cycles of PCR using pooled assays followed by exonuclease I treatment (New England Biolabs, MA, USA) to remove unincorporated primers. Final pre-amplified cDNA was diluted 1:5 in TE buffer. Gene expression analysis was carried out using the Biomark HD system from Fluidigm (Fluidigm Europe B.V.) in accordance with manufacturer's instructions and standard settings. Data was analyzed using the Real-Time PCR Analysis Software (Fluidigm Europe B.V.) and resulting CT values were normalized to Ppia to obtain dCT values.

Statistical Analysis

For treatment studies statistical analysis was performed using two-way ANOVA followed by Tukey post-hoc test. For comparison between groups, statistical testing was performed using non-parametric tests such as Mann-Whitney or Kruskal-Wallis followed by Dunn's multiple comparisons test. p values are indicated as *p<0.05, **p<0.01, ***p<0.001 and ****p<0.0001. All statistical analysis was performed with Graphpad Prism 7 for Mac.

### Results

### Example 2—the Omentum is a Critical Pre-Metastatic Niche for Ovarian Cancer Cells

The omentum is an adipose tissue formed from a fold of the peritoneal mesothelium. In humans the greater omentum covers the majority of the abdomen, whereas in mouse the omentum is only a thin stretch of adipose tissue located between the stomach, pancreas and spleen. The peritoneal spread of ovarian cancer can be modelled using the immortalized mouse ovarian epithelial cell line ID8 (Roby et al., 2000). The intra-peritoneal (i.p.) injection of ID8 cells leads to the development of diffuse peritoneal carcinomatosis and malignant ascites with a long latency period (up to 12 weeks). We used an ID8 cell line transduced to express firefly luciferase (ID8-luc) (Hagemann et al., 2008) to monitor tumor progression after i.p. injection by non-invasive bioluminescent imaging. We observed that ID8 cells localized primarily to the omentum for up to 35 days before spreading throughout the peritoneal cavity (data not shown). Infiltration of tumor cells into the omentum was confirmed by histological analysis, where visible tumor nodules could be observed within the adipose tissue from 35 days (data not shown). From day 63, the entire omentum was invaded by tumor cells and only residual adipose tissue remained (data not shown). Interestingly, despite the presence of obvious tumor nodules in omentum from day 35, accumulation of ascitic ID8 cells was not detectable until more than 50 days after i.p. injection (data not shown). Ultimately, ascites formation was associated the development of multiple distant metastases in the diaphragm and peritoneal wall (data not shown), reflecting late-stage disease in HGSOC patients. To establish the contribution of the omentum to the disease course in this model, we transplanted ID8-luc cells into omentectomized mice and monitored tumor growth. Whereas sham-operated and control mice showed a comparable course of tumor progression with accumulation of ascitic tumor cells after 63 days, we observed only minimal tumor growth in omentectomized mice and ascitic cells were barely detectable (data not shown).

### Conclusion

These data suggested the omentum was a critical pre-metastatic niche for tumor progression in this model and not merely a receptive site for peritoneal metastasis.

### Example 3—Ovarian Cancer Cells Colonize Fat-Associated Lymphoid Clusters in Close Contact with Omental Macrophages

The omentum has a particularly high density of fat-associated lymphoid clusters (FALC) that are thought to be important structures for capturing peritoneal antigens (Rangel-Moreno et al., 2009). Previous studies have proposed that FALC promote the colonization of omentum by ovarian cancer cells, however, neither B or T lymphocytes were shown to contribute to tumor growth (Clark et al., 2013). To visualize the localization of ID8 cells in the omentum, we labeled cells with Qdots (Qtracker®705) before i.p. injection and analyzed omentum by whole-mount confocal imaging. One day after injection, we observed that ID8 cells were located in the vicinity of FALCs, an area densely populated by omental macrophages (data not shown). To further characterize omental macrophages, we analyzed omentum by flow cytometry. Gating on the CD11b+ myeloid cell fraction (CD45.2+ Linneg (CDS, CD19, NK1.1, Ly6G, SiglecF) CD11b+) we identified macrophages as F4/80+CD64+ cells, whereas monocytes were F4/80− CD64− CCR2+(data not shown). To further characterize the heterogeneity of omental macrophages we analyzed expression of these markers on F4/80+ CD64+ cells. We observed a gradient of CD169 expression by omentum macrophages, where only CD169hi cells co-expressed Lyve1 (data not shown). Further analysis showed that CD169-Lyve1− and CD169int Lyve1− cells were also negative for CD163 and Tim4 expression (data not shown), indicating a less mature phenotype. In contrast, CD169hi Lyve1+ cells could be separated into four distinct populations based on CD163 and Tim4 expression; CD163+ Tim4+ (P1), CD163+ Tim4− (P2), CD163− Tim4− (P3) and CD163− Tim4+ (P4) (data not shown). Next, we analyzed the localization of these macrophage subsets in omentum by whole-mount confocal imaging. We found that CD169+ cells were distributed evenly throughout the tissue, whereas CD169+CD163+ cells were located at the interface between FALCs and the surrounding adipose tissue (data not shown). Tim4+ CD163− cells were mainly located within FALCs, whereas CD163+ Tim4+ cells were only found at the interface between FALCs and the surrounding adipose tissue (data not shown), similarly to CD169+CD163+ cells (data not shown). Therefore, P1 (CD163+ Tim4+) and P2 (CD163+ Tim4-) macrophages are specifically located in the tissue surrounding FALCs, whereas P3 (CD163− Tim4−) macrophages are widely distributed throughout the adipose tissue, and P4 (CD163− Tim4+) macrophages appear to be located within FALCs.

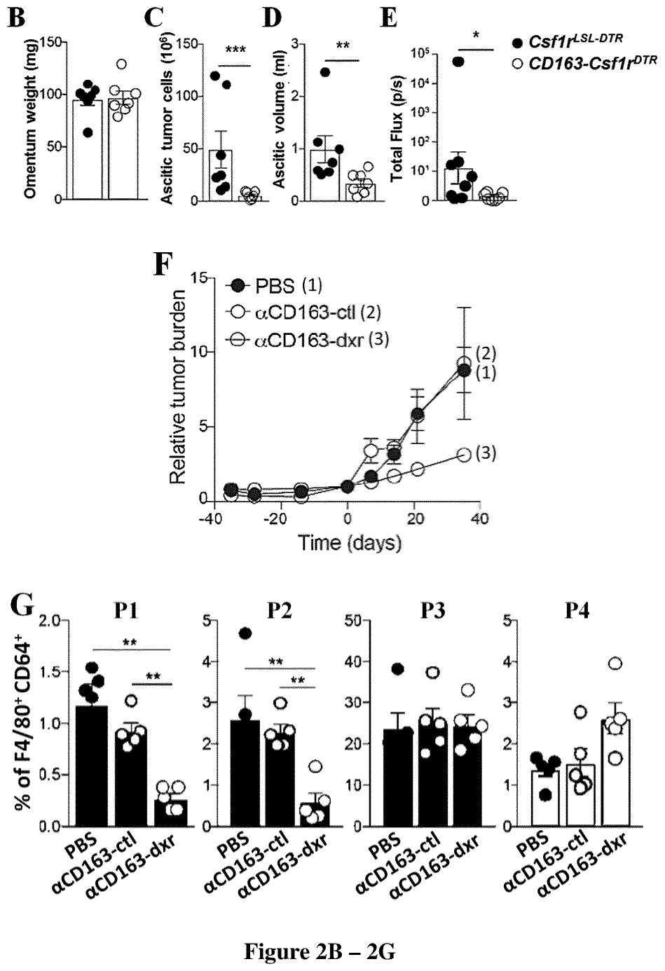

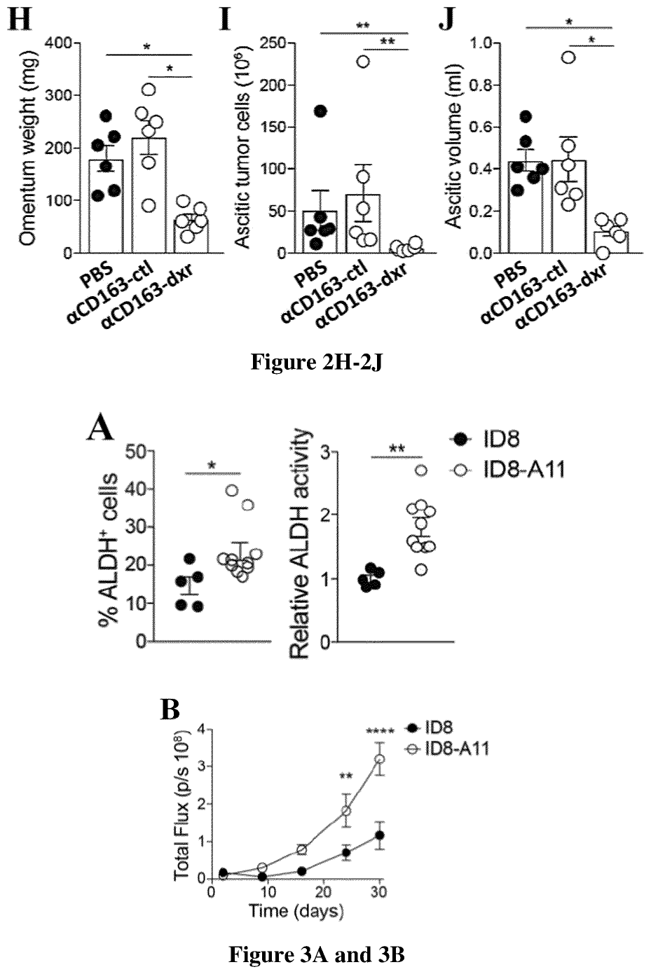

To determine the dynamics of these macrophage subsets during colonization of omentum by ovarian cancer cells and tumor progression, we performed a kinetic analysis by flow cytometry after transplantation of ID8 cells. Among the CD11b+ myeloid fraction, CCR2+ monocytes represent the most abundant population in naïve omentum, which remained unchanged for up to 4 weeks after ID8 cell injection (FIG. 2F). After 4 weeks, the proportion of monocytes decreased coincidently with an initial increase in CD16910 Lyve1− macrophages, followed by an increase in CD169int Lyve1− and CD169hi Lyve1+ cells after 8 weeks of tumor growth (data not shown). The proportional increase in CD169hi Lyve1+ macrophages appeared to be driven by an increase in P3 (CD163− Tim4−). In contrast, the proportion of P1 (CD163+ Tim4+), and to a lesser extent P2 (CD163+ Tim4−), was significantly decreased compared to normal omentum whereas P4 (CD163− Tim4+) remained stable (data not shown).

### Conclusion

In summary, these studies showed that ID8 cells colonize the omentum in the vicinity of FALCs and juxtaposed to resident CD163+ Tim4+ macrophages. As tumors progressed, there was an increase in the proportion of CD169+ Lyve1+ macrophages, but the fraction of resident CD163+ Tim4+ cells remained stable.

### Example 4—Embryonic Origin of Tissue-Resident CD163+ Tim4+ Macrophages in Omentum

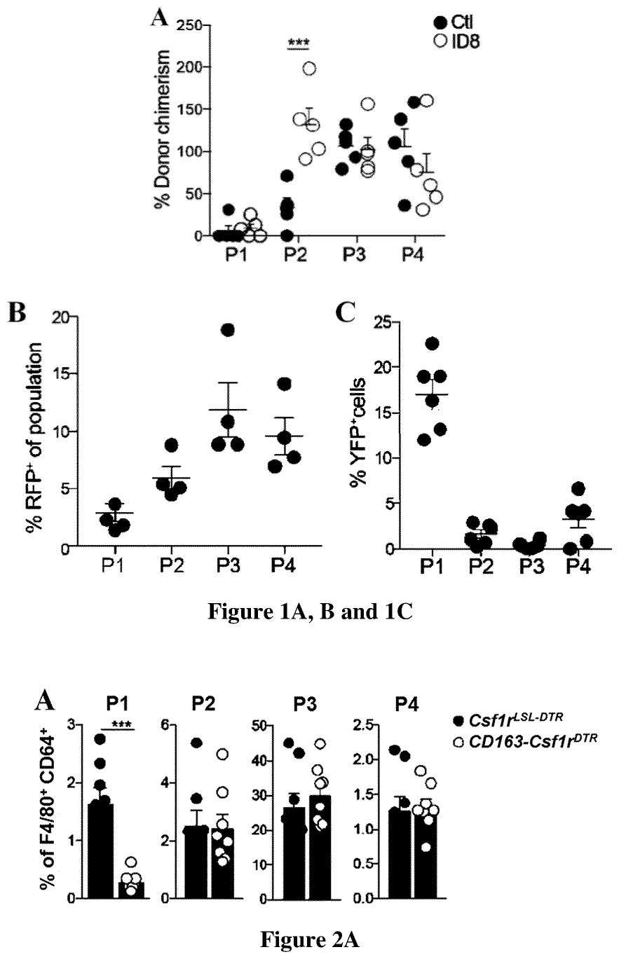

To evaluate the ontogeny and homeostasis of the different macrophage subsets in omentum during ID8 tumor development, we prepared protected radiation chimeras using CD45.1 congenic C57BL/6 recipient mice. The abdomen of mice was protected with lead shielding to avoid radiation-induced replacement of tissue-resident macrophages. Following irradiation, we adoptively transferred bone marrow cells from CD45.1×CD45.2 F1 mice (CD45.1/.2), which allowed the distinction between host (CD45.1+) and donor cells (CD45.1+/CD45.2+) by flow cytometry (data not shown). Five weeks after the bone marrow transplantation, chimerism was confirmed in blood by flow cytometry (data not shown) before mice were injected with ID8 cells. After an additional 8 weeks of tumor growth omentum was harvested for analysis. To track bone marrow-derived cells, CD45.1 and CD45.2 expression by omental macrophages was analyzed and normalized to blood monocytes. Chimerism within P3 (CD163− Tim4−) and P4 (CD163− Tim4+) populations was on average close to 100%, in both naïve and tumor-bearing mice (FIG. 1A). In contrast, chimerism in P1 (CD163+ Tim4+) macrophages was close to zero, irrespective of tumor growth, while the P2 (Tim4− CD163+) population showed approximately 40% chimerism in naïve mice and complete chimerism in tumor-bearing mice (FIG. 1A). These data showed that CD163+ Tim4+ omental macrophages (P1) were not replaced by bone marrow-derived cells throughout the course of this experiment, which was more than 3 months. However, P3 and P4 cells were completely replaced in both steady-state and during tumor development over this time course, suggesting these cells represent monocyte-derived macrophages of limited life-span. In contrast, CD163+ Tim4− macrophages (P2), were only partially replaced over 3 months at steady-state, although they showed complete replacement during tumor growth. Therefore, these cells represent long-lived monocyte-derived macrophages whose replacement is accelerated during tumor development.

To verify the contribution of circulating monocytes to omental TAM, we next performed a fluorescent fate-mapping experiment using mice expressing Cx3crl-CreERT2 (Cx3crlCreER) and a Rosa26-lox-STOP-lox(LSL)-tdRFP reporter allele (Cx3crl-R26tdRFP) (Goossens et al., 2019; Yona et al., 2013). In adult mice, Cx3cr1 is expressed by monocytes in blood and omentum, but expression is decreased as monocytes mature into macrophages (data not shown). To label monocytes during tumor development, Cx3crl-R26tdRFP mice were injected with ID8 cells and after 6 weeks mice were given a single dose of tamoxifen by oral gavage to activate RFP expression in Cx3cr1+ cells. 10 days later, RFP in omental macrophages was assessed by flow cytometry. As expected, P3 and P4 populations were most strongly labeled with RFP under these conditions, whereas P2 macrophages were labeled to a lesser extent (FIG. 1B). In contrast, there was minimal labeling of CD163+ Tim4+ cells (P1; FIG. 1B). These data are consistent with a rapid replacement of P3 and P4 macrophages by monocytes, while P2 cells are replaced with slightly slower kinetics. However, in keeping with data from shielded chimera experiments, there was very little replacement of P1 macrophages by Cx3Cr1-expressing precursors.

The experiments described above showed that CD163+ Tim4+ omental macrophages were not derived from bone marrow-dependent monocyte precursors, suggesting that these tissue-resident macrophages may be of embryonic origins. To evaluate the potential embryonic origins of these cells, we performed a fate mapping experiment with Cx3crlCreER mice in utero, since Cx3cr1 is expressed in both embryonic macrophage precursors and fetal monocytes (Yona et al., 2013). Cx3crl-R26YFP embryos were pulse-labeled with tamoxifen at E16.5 and YFP expression in macrophages from omentum was subsequently analyzed by flow cytometry at 8 weeks of age. After normalization of YFP+ cells to microglia to assess labeling efficiency (data not shown), we observed that approximately 20% of P1 macrophages (CD163+ Tim4+) in adult omentum were YFP+(FIG. 1C), indicating an embryonic origin for these cells. In contrast, very few YFP+ cells were detected in the P2 (CD163+ Tim4−), P3 (CD163− Tim4−) and P4 (CD163− Tim4−) populations (FIG. 1C), demonstrating that these cells were not derived from embryonic progenitors or were most likely replaced by monocyte-derived cells in the adult.

### Conclusion

Collectively, these experiments suggest that CD163+ Tim4+ macrophages in omentum are of embryonic origin and uniquely independent of bone marrow-derived monocytes, both in steady-state and during tumor growth and are thus likely to maintain themselves locally by self-renewal.

### Example 5—CD163+ Tim4+ Tissue-Resident Macrophages Express a Unique Transcriptional Profile

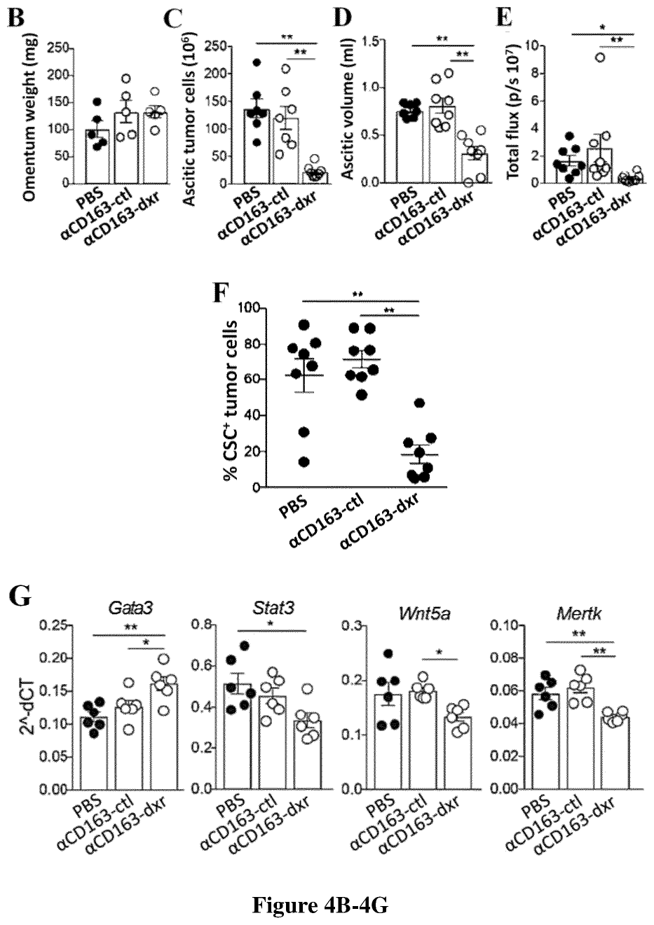

To evaluate the impact of tumor growth on the phenotype of different macrophage subsets in omentum, we performed transcriptional profiling of cells sorted by flow cytometry at steady-state and at different time points after seeding with tumor cells. We isolated P1, P2 and P3 macrophages by FACS from naïve omentum and at 5 or 10 weeks after injection of ID8 cells (data not shown). Sequencing libraries were prepared directly from snap-frozen cell pellets and sequenced to an average read depth of 42.7 million reads per sample. Expression values were normalized, filtered and analyzed for variations in gene expression using cqn and DeSeq2. A heatmap showing the 5000 most variable genes in the dataset is shown in figure S3. In order to analyze the relationship between the different macrophage populations, we performed a principal component analysis (PCA) combined with network analysis to show the n nearest neighbors. This analysis showed that the transcriptional profile of CD163+ Tim4+ resident macrophages (P1) did not undergo major changes during tumor growth, as all samples from this population clustered closely together (data not shown). Interestingly, the long-lived monocyte-derived CD163+ Tim4− macrophages (P2) were closely related to CD163+ Tim4+ resident macrophages (P1) at steady-state, but in tumor-bearing mice they became more closely aligned to P3 (data not shown), which likely reflects the increased replacement of P2 macrophages by monocyte-derived cells during tumor growth (data not shown). To further analyze the transcriptional changes between these three major subsets in established tumors, we extracted differentially expressed genes by pairwise and grouped comparisons of P1, P2 and P3 at 10 weeks of tumor growth (data not shown). We then performed a Self-Organizing-Map (SOM) clustering analysis to identify clusters of genes enriched in either a single population or a group of populations (data not shown). In the SOM clustering analysis, genes with a similar expression profile are first organized into SOMs. Within these maps, a pie chart depicts the relative enrichment of genes in the 3 populations analyzed (P1, P2 and P3) and the size of the pie slices reflects the gene enrichment. SOMs that are similar are then grouped together generating clusters of SOMs with a comparable gene enrichment profile. This analysis generated 16 different SOMs that were subsequently grouped in 7 distinct clusters. Cluster 2 was enriched in P1, cluster 6 in P2 and cluster 5 in P3. Cluster 3 contained genes enriched in both P1 and P2, and cluster 7 was enriched in both P2 and P3. We then used ClusterProfiler enrichment analysis and the Gene Ontology database (GO) for the different SOM clusters to identify biological processes (GO-BP) enriched in the different populations. This analysis revealed multiple enriched processes in clusters 2, 3, 5 and 6, the 15 most significant terms are shown in FIG. 4E. Of particular interest, positive regulation of the JAK-STAT signaling was uniquely enriched in CD163+ Tim4+ macrophages (data not shown), whereas pathways related to angiogenesis, blood vessel development and tissue remodeling were shared between CD163+ populations (data not shown). Interestingly, the STAT pathway is part of the self-renewal gene regulatory network in macrophages (Soucie et al., 2016), in line with the ability of these cells to maintain themselves independently of bone-marrow derived monocytes. In addition, the pathways and processes enriched in both CD163+ populations (P1 and P2) have previously been linked with tumor-promoting functions of TAM (Noy and Pollard, 2014). In contrast, pathways associated with T cell differentiation were uniquely associated with P3 (data not shown).

### Conclusion

This analysis suggests a functional diversification of omental macrophage subsets in the context of tumor growth.

### Example 6—Specific Depletion of CD163+ Tim4+ Macrophages Prevents Metastatic Spread of Ovarian Cancer

Next, we sought to analyze the specific contributions of omental macrophage subsets to disease progression in our model. Initial experiments showed that tumor development and accumulation of malignant ascites were not affected in Ccr2−/− mice (data not shown). These mice have impaired recruitment of monocyte-derived cells, suggesting a redundant function of monocyte-derived macrophages in disease progression. To assess the specific contribution of CD163+ Tim4+ tissue-resident macrophages (P1), we generated transgenic mice that exclusively express DTR in CD163+ macrophages (Cd163-Csf1rDTR); Cd163-iCre knock-in mice (Cd163iCre) were crossed with transgenic mice expressing a LSL-DTR cassette under control of the Csflr-promoter (Tg(Csflr-LSL-DTR)). Flow cytometry analysis of omentum from Cd163-CsflrDTR mice 24 h after a single injection of diptheria toxin (DT; 4 ng/kg) confirmed the specific ablation of CD163+P1 and P2 macrophages (data not shown). However, 6 days after DT treatment, monocyte-derived P2 macrophages were completely restored while resident P1 macrophages remained absent (data not shown). Thus, this approach allows the specific ablation CD163+P1 macrophages and the opportunity to assess their unique contribution towards tumor progression. We treated cohorts of Cd163-Csf1rDTR and Cre-negative littermate controls (Csf1rLSL-DTR) with DT and 6 days later injected ID8 cells i.p. After 10 weeks, omentum was collected for analysis by flow cytometry and tumor progression assessed. At this time point, P1 macrophages remained depleted in omenta from Cd163-Csf1rDTR mice while all other populations were unchanged (FIG. 2A). Interestingly, although tumor seeding of the omentum was unaffected (FIG. 2B), Cd163-Csf1rDTR mice showed a significant reduction in ascitic tumor cells (FIG. 2C) and reduced ascites (FIG. 2D). Moreover, Cd163-Csf1rDTR mice had significantly reduced metastases to the diaphragm and other peritoneal organs (FIG. 2E).

### Conclusion

These data suggest tissue-resident CD163+ Tim4+ macrophages in the omentum contribute significantly to the metastatic spread of ovarian cancer cells and the development of invasive disease.

### Example 7—the Role of CD163+ Macrophages in Tumor Progression

To further substantiate the role of CD163+ macrophages in tumor progression, we used CD163-targeted cytotoxic lipid nanoparticles (LNPs) to therapeutically deplete CD163+ macrophages in tumor-bearing mice (Etzerodt et al. 2019. The Journal of experimental medicine 216(10), 2394-2411.). These LNPs contain 5% polyethylene glycol (PEG; 2000 mw) in the lipid bilayer that minimizes non-specific phagocytic uptake and are loaded with the cytotoxic drug doxorubicin (dxr) to kill target cells. LNPs are targeted specifically to CD163-expressing cells by conjugation of an anti-CD163 monoclonal antibody to the PEG (Etzerodt et al., 2012). To achieve therapeutic depletion of CD163+ macrophages, mice were injected with ID8 cells and 5 weeks later treated intravenously twice a week for 5 weeks with either vehicle, empty CD163-targeted LNPs (αCD163-ctrl) or dxr-loaded LNPs (αCD163-dxr). In vivo bioluminescent imaging showed that continuous depletion of CD163+ cells after αCD163-dxr treatment, resulted in a significant reduction of overall tumor burden in the abdomen (FIG. 2F). As expected, sustained depletion of CD163+ cells led to the specific loss of both P1 and P2 macrophages in omentum (FIG. 2G), which was accompanied by a significant decrease of tumor burden in both omentum and ascites (FIG. 2H-J).

### Conclusion

The data suggest that the absence of monocyte-derived CD163+ macrophages (P2) contribute to reduced tumor growth in the omentum (FIG. 2H), which was not observed in the specific absence of tissue-resident CD163+ Tim4+ macrophages (P1) (FIG. 2B).

### Example 8—Ovarian Cancer Cells in Ascites Acquire Cancer Stem Cell (CSC) Characteristics

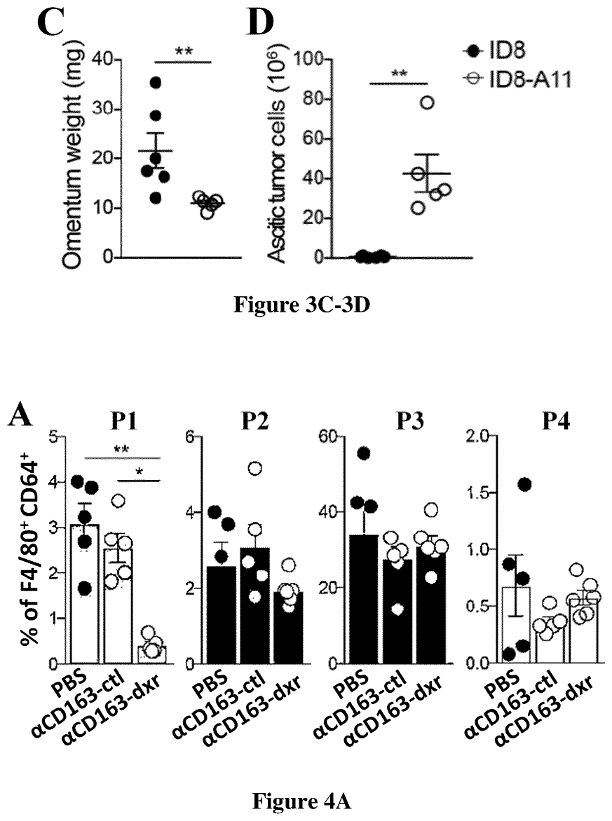

Ascitic tumor development and peritoneal metastases are characteristic of HGSOC and indicate a particularly poor prognosis. Our studies showed a long latency in ascitic tumor development and peritoneal spread of ID8 cells after seeding the omentum, and omentectomy prevented development of ascitic disease, suggesting the omentum represents a key pre-metastatic niche. In addition, the studies described above showed that tissue-resident macrophages in omentum contributed significantly to the accumulation of ascitic cells and the development of invasive disease. To further explore the invasive phenotype of ascitic ID8 cells and define the mechanisms behind this invasive transition, we performed transcriptomic analysis of cultured ID8 cells and tumor cells isolated from malignant ascites 11 weeks after transplantation (ID8-A11). With this analysis we found clusters of GO terms up-regulated in ID8-A11 cells that represent biological processes often associated with metastatic tumor cells, including; drug metabolism, epithelial cell migration, organization of cell junctions and organ development (data not shown). In contrast, clusters of GO terms that were down-regulated in ID8-A11 cells were mainly linked with biological processes associated with cell division such as cytokinesis, cell cycle, DNA repair and replication (data not shown). To further explore these pathways, we performed geneset enrichment analysis (GSEA); in accordance with a metastatic phenotype, GSEA showed a positive enrichment of genesets associated with epithelial to mesenchymal transition (EMT) and down-regulation of genes associated with cell cycle and cell division (data not shown). The inverse enrichment of EMT pathways versus cell division, coupled with the up-regulation of WNT, NOTCH and STAT3 signaling (data not shown), suggested ID8-A11 cells may have acquired a cancer stem cell (CSC)-like phenotype. Ascitic tumor cells with CSC characteristics have been associated with advanced human ovarian cancer, where they form multilayered spheroid structures (Bapat et al., 2005). To assess the CSC-like phenotype of ascitic ID8 cells, we compiled a list of genes that were reported as biomarkers of ovarian CSCs and analyzed their expression in ID8-A11 versus ID8 cells. This analysis confirmed an up-regulation of CSC markers in ID8-A11 cells (FIG. 3A). In addition, we established a panel of CSC cell-surface markers and measured their expression by flow cytometry. In agreement with gene expression analysis, ID8-A11 cells showed increased expression of a number of surface markers for CSCs including CD44, CD54, CD55, CD106 and CD117 (data not shown). As mentioned above, spheroid formation is a functional characteristic of CSCs, as is the increased activity aldehyde dehydrogenase (ALDH) (Kim et al., 2018). In contrast to cultured ID8 cells, ascitic ID8-A11 cells rapidly formed spheroids in vitro (data not shown). In addition, flow cytometry analysis of ALDH activity using the ALDEFLUOR assay showed increased ALDH activity in ID8-A11 cells (data not shown). Finally, acquisition of CSC characteristics is associated with increased tumor-initiating potential and metastatic spread. We therefore injected cohorts of mice with either ID8 or ID8-A11 cells and followed tumor burden and spread in vivo using bioluminescence. The overall tumor burden in mice injected with ID8-A11 cells was already significantly increased after 20 days, whereas parental ID8 cells still had not established significant tumors (FIG. 3B). Moreover, when tumors did establish in mice injected with ID8 cells, these were restricted to the omentum whereas ID8-A11 cells had spread throughout the peritoneal cavity (data not shown). This was further substantiated in ex vivo analysis that showed ID8 cells were restricted to the omentum at this time point and not present in ascites, whereas ID8-A11 cells generated considerable ascitic tumor growth (FIG. 3C&3D).

### Conclusion

These studies demonstrate that the transition to invasive disease in this model was associated with the acquisition of CSC characteristics by ascitic tumor cells.

### Example 9—CD163+ Tim4+ Tissue-Resident Macrophages Promote the CSC-Like Phenotype Ovarian Cancer Cells

We next evaluated the impact of tissue-resident macrophages in omentum on the acquisition of CSC characteristics by ascitic ID8 cells. CD163+ macrophages in the omentum were depleted by 3 consecutive injections of CD163-targeted cytotoxic LNPs (αCD163-dxr) on days 1, 3, and 5, control mice were injected with either vehicle or empty LNPs (αCD163-ctrl). Monocyte-derived CD163+ macrophages (P2) were then allowed to recover before injection of ID8 cells on day 8 and the development of invasive disease was analysed at 10 weeks. Specific depletion of resident CD163+ Tim4+ macrophages (PO in omentum was confirmed by flow cytometry (FIG. 4A), as was the impaired development of invasive disease, including the number of ascitic tumor cells and peritoneal metastases (FIG. 4B-E). To evaluate the impact on CSC-phenotype in ID8 cells, we analyzed the expression of a panel of CSC markers by flow cytometry on ascitic ID8 cells from control treated mice and after depletion of resident omental macrophages. We then conducted an unsupervised gating analysis and dimensionality reduction using t-distributed Stochastic Neighbor Embedding (tSNE). Samples corresponding to individual treatment groups were subsequently gated out and re-plotted as individual tSNE plots (data not shown). This analysis showed that ascitic tumor cells from αCD163-dxr treated mice, specifically lacking resident CD163+ Tim4+ macrophages in the omentum, clearly separated from ascitic tumor cells in control mice (data not shown). Subsequent color mapping of the staining intensity for each of the CSC markers revealed a loss of tumor cells expressing CD54, CD55, CD106 and CD117 in αCD163-dxr treated mice (data not shown). The reduced frequency of CD54, CD55, CD106 and CD117 expressing cells was subsequently confirmed by manual gating. This showed that in control mice >60% of ascitic ID8 cells expressed CSC markers, which was reduced to less than 20% upon depletion of resident CD163+ Tim4+ macrophages in the omentum (FIG. 4F). Thus, the specific depletion of resident omental macrophages resulted in reduced accumulation of ascitic tumor cells with a CSC-like phenotype. The acquisition of CSC characteristics is frequently associated with EMT (Nieto et al., 2016) and our previous transcriptomic analysis of ascitic ID8 cells showed an up-regulation of EMT associated genes. To confirm this data, we analyzed the expression of transcription factors (TFs) known to drive either EMT or the reverse process known as mesenchymal to epithelial transition (MET) in ID8-A11 cells. This analysis confirmed an increased expression of TFs driving EMT (e.g., Zeb2), whereas TFs involved in MET were downregulated (e.g., Gata3) (data not shown). Interestingly, Gata3 alone was recently shown to be sufficient to reverse EMT and inhibit metastases in breast and colon cancer (Yan et al., 2010; Z. Yang et al., 2017). Since EMT has been suggested to precede the acquisition of CSC characteristics, we evaluated the expression of EMT/MET regulators in ID8 cells from omentum after depletion of CD163+ Tim4+ resident macrophages. Mice were treated as described above and ID8 tumor cells were isolated from the omentum and expression of EMT and MET associated TFs was analyzed. We observed a significant increase in expression of Gata3 in ID8 cells from omentum after depletion of resident macrophages, which was accompanied by decreased expression Stat3, Wnt5a and Mertk (FIG. 4G), all positive regulators of EMT and CSC phenotype.

### Conclusion

These experiments show that the acquisition of EMT and CSC characteristics by ID8 cells, and the development of invasive disease, is promoted by the resident CD163+ Tim4+ macrophages in the omentum.

### Example 10—Treatment of Pancreatic Ductal Adenocarcinoma

### Aim of Study

To further support the specific importance of CD163⁺ TAM in tumour progression, a CD163⁺ TAM depletion study in a GEMM model of pancreatic ductal adenocarcinoma (PDAC mice: P48-Cre; LSL-Kras^{G12D}; LSL− Trp53^{R172H}) was conducted.

### Materials, Methods and Results

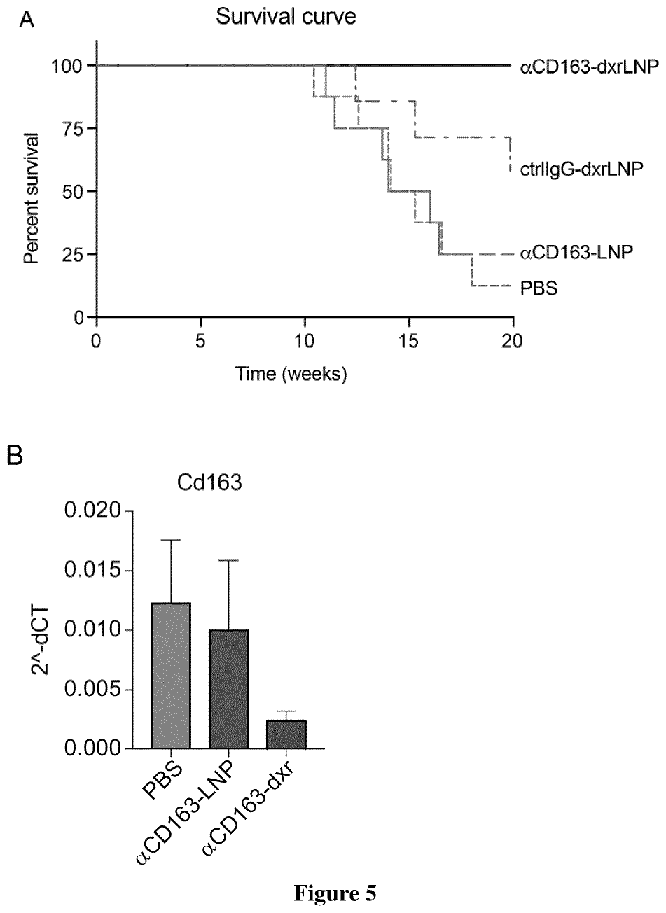

PDAC mice were treated with αCD163-dxr (1 mg/kg dxr) twice a week from 6 weeks of age; CD163⁺ TAM depletion resulted in increased survival with all mice still being alive at 20 weeks of age (FIG. 5). In contrast, survival of control mice receiving either vehicle (PBS) or empty liposomes (αCD163-LNP) started to decrease from 10 weeks of age, with less than 25% survival at 20 weeks.

### Conclusion

This example shows that pancreatic ductal adenocarcinoma can be treated and/or alleviated using a αCD163-dxr antibody. Thus, pancreatic cancer is indeed a target for the compositions, combinations and kits according to the invention.

### Example 11—In Vitro Analysis of Duocarmycin Conjugated Anti-Mouse Tim4 Cytotoxicity

### Aim of Study

To assess the in vitro cytotoxicity of duocarmycin conjugated anti-mouse Tim4.

### Materials and Methods

### Generation of Mouse Tim4 Expressing Cell Line:

cDNA coding for mouse Tim4 (NM 178759.4) was obtained from genscript and inserted in pcDNA5/FRT using HindIII and BamHI restriction sites. To generate a stable cell line expressing mouse Tim4, HEK FlpIn293 cells were co-transfected with pOG44 plasmid and Tim4_pcDNA/FRT plasmid and selected for Tim4 expression using 150 μg/ml Hygromycin. Stable expression of mouse Tim 4 was subsequent verified using western blotting against mouse Tim4 and image cytometry analysis.

### Preparation of Anti-Mouse Tim4-Duocarmycin:

Anti-mouse Tim4 antibody (clone RMT4-54, BioXCell) or control IgG (rat IgG 2A, clone 2A3, BioXCell) was conjugated with OSu-PEG4-vc-PAB-Duocarmycin SA (Creative Biolabs) in a ratio of antibody to drug of 1:6 and a final antibody concentration of 1 mg/ml. pH was adjusted to 8.5 using Borate buffered saline pH 8.5 and reaction was left over night at 4° C. Non-conjugated duocarmycin was subsequently removed by dialysis overnight against 1×PBS at 4° C. resulting in an anti-mouse Tim4 antibody-drug conjugate with a DAR of 4.5 (0.86 mg/ml Tim4, 30.2 μM duocarmycin) and control IgG antibody-drug conjugate with a DAR of 4.2 (0.6 mg/ml antibody, 17 μM duocarmycin).

### Results

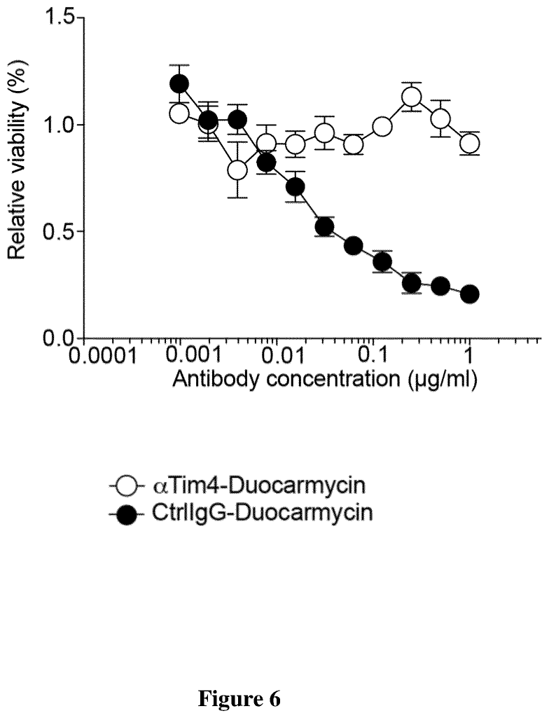

FlpIn293 cells expressing mouse Tim4 was seeded in black clear bottom 96 well plates and incubated for 24 hrs. Following, 2 fold dilution (1 ug/ml to 1 ng/ml, each n=4) of duocarmycin conjugated anti-mouse Tim4 or control IgG was added to the cells. After 48 hrs, viability was measured using a calcein-AM based viability assay and relative viability was calculated using cells not receiving duocarmycin conjugated antibody (FIG. 6).

### Conclusion

Anti-mouse Tim4-duocarmycin could efficiently kill the Tim4 expressing cells in vitro.

### Example 12—In Vivo Analysis of Duocarmycin Conjugated Anti-Mouse Tim4 Cytotoxicity and Specificity

### Aim of Study

To assess the in vivo cytotoxicity of duocarmycin conjugated anti-mouse Tim4.

Materials and Methods

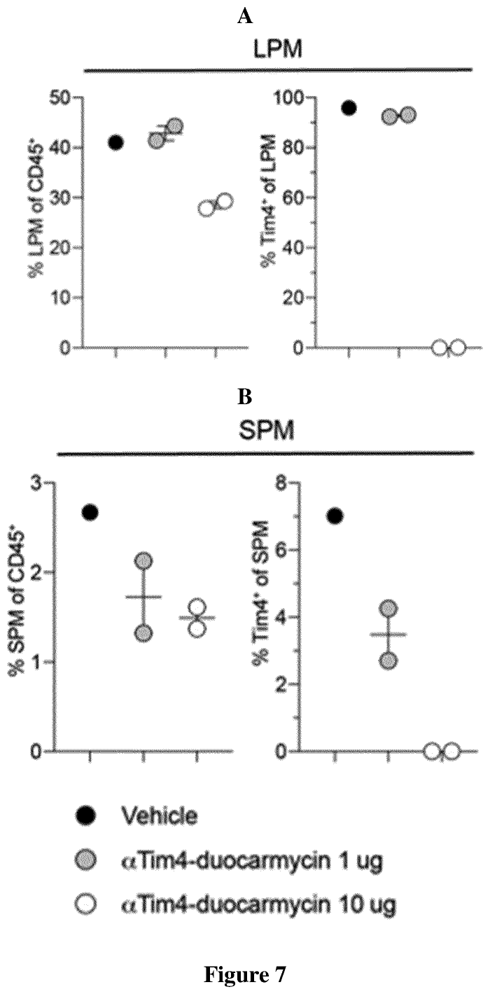

Naïve C57B1/6j were injected i.p with either vehicle (1×PBS, n=1) or 1 μg duocarmycin conjugated anti-mouse Tim4 (n=2) or 10 μg duocarmycin conjugated anti-mouse Tim4 (n=2). After 24 hrs mice were euthanized and peritoneal immune cells were harvested by peritoneal lavage using 1×PBS supplemented with 2 mM EDTA. Tim4 is mainly expressed by large peritoneal macrophages (LPM) and to assess cytotoxicity and specificity of duocarmycin conjugated anti-mouse Tim4 antibody, composition peritoneal immune cells were analyzed using flow cytometry. Peritoneal macrophages were gated as Live cells, CD45.2⁺, Ling^{neg} (CD5, CD19, Ly6G, NK1.1), CD11⁺ and subsequently large peritoneal macrophages (LPM) were identified as F4/80⁺ MHCII⁺ whereas small peritoneal macrophages (SPM) were identified as F4/80⁻ MHCII⁺ (data not shown). Subsequently frequency of Tim4 positive LPM and SPM were analyzed (FIG. 7).

### Results

The data in FIG. 7 shows that the Tim4 antibody depletes Tim4 in vivo in both the large peritoneal macrophages (LPM) (FIG. 7A) and the small peritoneal macrophages (SPM) (FIG. 7B).

### Conclusion

This example confirms that an anti-mouse Tim4-duocarmycin antibody is specific and can efficiently deplete Tim4 expressing macrophages in vivo.

### Discussion

The vast majority of cancer-related deaths are due to the development of metastatic disease. The metastatic spread of cancer can be described according to two basic models: The predominant linear model, dictates a step-wise progression of primary tumors before dissemination of fully metastatic malignant cells. Whereas the parallel model, accounts for the early dissemination of cancer cells and the formation of distant metastases that develop in parallel with the primary tumor. While there has been extensive research into the step-wise progression of primary tumors towards a metastatic phenotype, relatively little is known about the role of cells that form the pre-metastatic niche for disseminated cancer cells and their involvement in the metastatic spread of disease. Given that macrophages populate all adult tissues and the proven role of TAM in promoting invasion and metastasis in experimental models (Noy and Pollard, 2014), it's likely that resident macrophages form an important component of the tissue niche for the malignant progression of disseminated cancer cells.

Here we studied the origins and function of macrophages in a model of metastatic ovarian cancer using a novel intersectional transgenic approach and antibody-targeted cytotoxic liposomes to specifically deplete CD163+ macrophages. We describe how tissue-resident CD163+ macrophages in omentum play a specific role in the malignant progression of ovarian cancer and the development of invasive disease. We showed that ovarian cancer cells injected into the peritoneal cavity infiltrate the omentum in the vicinity of fat-associated lymphoid-clusters (FALC) and in close contact with resident CD163+ macrophages. Using flow cytometry and whole-mount imaging, we identified 4 distinct subsets of CD169+ Lyve-1+ mature macrophages in omentum based on their expression of CD163 and Tim4. CD163 has been used extensively as a marker of tissue macrophages in humans, where the frequency of CD163+ TAM shows a striking correlation with poor clinical outcome in a range of cancers (Komohara et al., 2014). Conversely, few studies have addressed the expression of Tim4 in human macrophages. Recent studies have shown Tim4 is expressed by a range of long-lived tissue-resident macrophages in the mouse, including Kupffer cells (Scott et al., 2016), large peritoneal macrophages (LPM) (Rosas et al., 2014), gut and cardiac macrophages (De Schepper et al., 2018; S. A. Dick et al., 2019), and is thus emerging as a marker of tissue-resident macrophages with potential for self-renewal. When analyzing the ontogeny of CD169+ Lyve-1+ macrophages in omentum, we found that Tim4 expression alone did not distinguish long-lived cells, since Tim4+ CD163− macrophages (P4), were monocyte-derived and rapidly replaced by bone marrow-derived cells. Whereas, CD163+ Tim4+ (P1) macrophages were of embryonic origin and uniquely independent of bone marrow-derived monocytes in the adult, in both steady-state and during tumor development. In contrast, CD163+ Tim4− macrophages (P2) were relatively long-lived at steady-state, compared to CD163-negative cells, but were quickly replaced by monocyte-derived cells after tumor initiation. Transcriptomic analysis revealed a close relationship between CD163+ P1 and P2 macrophages at steady-state, whereas, during tumor development monocyte-derived Tim4-negative P2 macrophages significantly diverged from Tim4+ P1 cells and became more similar to short-lived CD163-negative monocyte-derived cells (P3). In fact, the phenotype of CD163+ Tim4+ resident macrophages in omentum was remarkably stable during tumor progression, suggesting these cells maintain a level of tissue imprinting that is lost from more short-lived cells. Prophylactic depletion of CD163+ macrophages, allowing recovery of monocyte-derived CD163+ Tim4− cells, revealed an important and specific role for resident CD163+ Tim4+ omental macrophages in the development of invasive disease in this model. Furthermore, the therapeutic depletion of CD163+ TAM using cytotoxic liposomes had a major effect on tumor progression—illustrating the potential therapeutic implications for targeting TAM subsets in ovarian cancer.

Macrophages play important roles in organogenesis and tissue remodeling (Pollard, 2009) and have been shown to affect epithelial cell plasticity and stimulate tissue stem cells (Chakrabarti et al., 2018; Lee et al., 2018). The acquisition of stem-like characteristics by cancer cells (cancer stem cells; CSC) has been suggested to promote tumor progression and metastasis (Kreso and J. E. Dick, 2014). CSCs show increased anchorage-independent survival and high levels of resistance to chemotherapy or radiotherapy and thus have major implications for disease recurrence from disseminated tumor cells. EMT is also frequently associated with CSCs and accounts for the acquisition of migratory and invasive properties (Nieto et al., 2016). Several reports have shown that ascitic tumor cells from late-stage ovarian cancer patients show CSC-like characteristics (Bapat et al., 2005; Michela Lupia, 2017), which may explain the rapid progression of late-stage disease in these patients and the high frequency of disease recurrence. When analyzing the transcriptome of ascitic tumor cells in our model, we found a significant enrichment of pathways associated with CSCs and EMT. Interestingly, not only did the specific depletion of CD163+ Tim4+ resident macrophages in omentum decrease the formation of malignant ascites, it also reduced the frequency of CSCs among the few ascitic tumor cell that did accumulate. Analysis of tumor cells in the omentum showed that genes associated with regulation of EMT and CSCs, namely Stat3 (Abubaker et al., 2014), Wnt5a (Ford et al., 2014) and Mertk (Jung et al., 2016), were downregulated in tumor cells when CD163+ Tim4+ macrophages were absent, whereas Gata3, a negative regulator of EMT, was upregulated. In this regard, it is of particular interest that clustering analysis showed CD163+ Tim4+ macrophages were enriched for genes associated with positive regulation of the JAK-STAT pathway.

In summary, our data show that tissue-resident macrophages in omentum play a specific role in the malignant progression of disseminated tumor cells and the development of invasive disease in a mouse model of metastatic ovarian cancer. These studies add significantly to our understanding of TAM heterogeneity and the specific contribution of different macrophage subsets to disease progression. The axes of interaction between tissue-resident macrophages and cancer cells could represent important new therapeutic targets, not only in ovarian cancer but also other cancers where the development of CSCs can have a disastrous impact on disease prognosis.

### REFERENCES

Throughout this application, various references describe the state of the art to which this invention pertains. The disclosures of these references are hereby incorporated by reference into the present disclosure.
Abubaker, K., Luwor, R. B., Zhu, H., McNally, O., Quinn, M. A., Burns, C. J., Thompson, E. W., Findlay, J. K., Ahmed, N., 2014. Inhibition of the JAK2/STAT3 pathway in ovarian cancer results in the loss of cancer stem cell-like characteristics and a reduced tumor burden. BMC Cancer 14, 317. doi:10.1186/1471-2407-14-317
Bapat, S. A., Mali, A. M., Koppikar, C. B., Kurrey, N. K., 2005. Stem and progenitor-like cells contribute to the aggressive behavior of human epithelial ovarian cancer. Cancer Res 65, 3025-3029. doi:10.1158/0008-5472.CAN-04-3931
Baratin, M., Simon, L., Jorquera, A., Ghigo, C., Dembele, D., Nowak, J., Gentek, R., Wienert, S., Klauschen, F., Malissen, B., Dalod, M., Bajenoff, M., 2017. T Cell Zone Resident Macrophages Silently Dispose of Apoptotic Cells in the Lymph Node. Immunity 47, 349-362.e5. doi:10.1016/j.immuni.2017.07.019
Bénézech, C., Luu, N.-T., Walker, J. A., Kruglov, A. A., Loo, Y., Nakamura, K., Zhang, Y., Nayar, S., Jones, L. H., Flores-Langarica, A., McIntosh, A., Marshall, J., Barone, F., Besra, G., Miles, K., Allen, J. E., Gray, M., Kollias, G., Cunningham, A. F., Withers, D. R., Toellner, K. M., Jones, N. D., Veldhoen, M., Nedospasov, S. A., McKenzie, A. N. J., Caamalio, J. H., 2015. Inflammation-induced formation of fat-associated lymphoid clusters. Nature Immunology 16, 819-828. doi:10.1038/ni.3215
Bushnell, B., 2014. BBMap: A Fast, Accurate, Splice-Aware Aligner. https://www.osti.gov/servlets/purl/1241166
Chakarov, S., Lim, H. Y., Tan, L., Lim, S. Y., See, P., Lum, J., Zhang, X.-M., Foo, S., Nakamizo, S., Duan, K., Kong, W. T., Gentek, R., Balachander, A., Carbajo, D., Bleriot, C., Malleret, B., Tam, J. K. C., Baig, S., Shabeer, M., Toh, S.-A. E. S., Schlitzer, A., Larbi, A., Marichal, T., Malissen, B., Chen, J., Poidinger, M., Kabashima, K., Bajenoff, M., Ng, L. G., Angeli, V., Ginhoux, F., 2019. Two distinct interstitial macrophage populations coexist across tissues in specific subtissular niches. Science 363. doi:10.1126/science.aau0964
Chakrabarti, R., Celia-Terrassa, T., Kumar, S., Hang, X., Wei, Y., Choudhury, A., Hwang, J., Peng, J., Nixon, B., Grady, J. J., DeCoste, C., Gao, J., van Es, J. H., Li, M. O., Aifantis, I., Clevers, H., Kang, Y., 2018. Notch ligand D111 mediates cross-talk between mammary stem cells and the macrophageal niche. Science 127, eaan4153. doi:10.1126/science.aan4153
Clark, R., Krishnan, V., Schoof, M., Rodriguez, I., Theriault, B., Chekmareva, M., Rinker-Schaeffer, C., 2013. Milky Spots Promote Ovarian Cancer Metastatic Colonization of Peritoneal Adipose in Experimental Models. Am. J. Pathol. 183, 576-591. doi:10.1016/j.ajpath.2013.04.023
De Schepper, S., Verheijden, S., Aguilera-Lizarraga, J., Viola, M. F., Boesmans, W., Stakenborg, N., Voytyuk, I., Schmidt, I., Boeckx, B., Dierckx de Casterle, I., Baekelandt, V., Gonzalez Dominguez, E., Mack, M., Depoortere, I., De Strooper, B., Sprangers, B., Himmelreich, U., Soenen, S., Guilliams, M., Vanden Berghe, P., Jones, E., Lambrechts, D., Boeckxstaens, G., 2018. Self-Maintaining Gut Macrophages Are Essential for Intestinal Homeostasis. Cell 175, 400-415.e13. doi:10.1016/j.ce11.2018.07.048
Dick, S. A., Macklin, J. A., Nejat, S., Momen, A., Clemente-Casares, X., Althagafi, M. G., Chen, J., Kantores, C., Hosseinzadeh, S., Aronoff, L., Wong, A., Zaman, R., Barbu, I., Besla, R., Lavine, K. J., Razani, B., Ginhoux, F., Husain, M., Cybulsky, M. I., Robbins, C. S., Epelman, S., 2019. Self-renewing resident cardiac macrophages limit adverse remodeling following myocardial infarction. Nature Immunology 20, 29-39. doi:10.1038/s41590-018-0272-2
Dobin, A., Davis, C. A., Schlesinger, F., Drenkow, J., Zaleski, C., Jha, S., Batut, P., Chaisson, M., Gingeras, T. R., 2012. STAR: ultrafast universal RNA-seq aligner. Bioinformatics 29, 15-21. doi:10.1093/bioinformatics/bts635
Etzerodt, A., Kjolby, M., Nielsen, M. J., Maniecki, M., Svendsen, P., Moestrup, S. K., 2013. Plasma Clearance of Hemoglobin and Haptoglobin in Mice and Effect of CD163 Gene Targeting Disruption. Antioxid Redox Signal 18, 2254-2263. doi:10.1089/ars.2012.4605
Etzerodt, A., Maniecki, M. B., Graversen, J. H., Moller, H. J., Torchilin, V. P., Moestrup, S. K., 2012. Efficient intracellular drug-targeting of macrophages using stealth liposomes directed to the hemoglobin scavenger receptor CD163. J Control Release 160, 72-80. doi:10.1016/j.jconre1.2012.01.034
Etzerodt, A., Moestrup, S. K., 2013. CD163 and inflammation: biological, diagnostic, and therapeutic aspects. Antioxid. Redox Signal. 18, 2352-2363. doi:10.1089/ars.2012.4834
Ford, C. E., Punnia-Moorthy, G., Henry, C. E., Llamosas, E., Nixdorf, S., Olivier, J., Caduff, R., Ward, R. L., Heinzelmann-Schwarz, V., 2014. The non-canonical Wnt ligand, Wnt5a, is upregulated and associated with epithelial to mesenchymal transition in epithelial ovarian cancer. Gynecologic Oncology 134, 338-345. doi:10.1016/j.ygyno.2014.06.004
Fritze, A., Hens, F., Kimpfler, A., Schubert, R., Peschka-Suss, R., 2006. Remote loading of doxorubicin into liposomes driven by a transmembrane phosphate gradient. Biochim Biophys Acta 1758, 1633-1640. doi:10.1016/j.bbamem.2006.05.028
Geissmann, F., Manz, M. G., Jung, S., Sieweke, M. H., Merad, M., Ley, K., 2010. Development of monocytes, macrophages, and dendritic cells. Science 327, 656-661. doi:10.1126/science.1178331
Ginhoux, F., Guilliams, M., 2016. Tissue-Resident Macrophage Ontogeny and Homeostasis. Immunity 44, 439-449. doi:10.1016/j.immuni.2016.02.024
Goossens, P., Rodriguez-Vita, J., Etzerodt, A., Masse, M., Rastoin, O., Gouirand, V., Ulas, T., Papantonopoulou, O., Van Eck, M., Auphan-Anezin, N., Bebien, M., Verthuy, C., Vu Manh, T. P., Turner, M., Dalod, M., Schultze, J. L., Lawrence, T., 2019. Membrane Cholesterol Efflux Drives Tumor-Associated Macrophage Reprogramming and Tumor Progression. Cell Metabolism. doi:10.1016/j.cmet.2019.02.016
Gray, E. E., Cyster, J. G., 2012. Lymph node macrophages. J Innate Immun 4, 424-436. doi: 10.1159/000337007
Hagemann, T., Lawrence, T., McNeish, I., Charles, K. A., Kulbe, H., Thompson, R. G., Robinson, S. C., Balkwill, F. R., 2008. “Re-educating” tumor-associated macrophages by targeting NF-kappaB. J. Exp. Med. 205, 1261-1268. doi:10.1084/jem.20080108
Hagiwara, A., Takahashi, T., Sawai, K., Taniguchi, H., Shimotsuma, M., Okano, S., Sakakura, C., Tsujimoto, H., Osaki, K., Sasaki, S., 1993. Milky spots as the implantation site for malignant cells in peritoneal dissemination in mice. Cancer Res 53, 687-692.
Hansen, K. D., Irizarry, R. A., Wu, Z., 2012. Removing technical variability in RNA-seq data using conditional quantile normalization. Biostatistics 13, 204-216. doi:10.1093/biostatistics/locr054
Ferlay J, Ervik M, Lam F, Colombet M, Mery L, Piñeros M, Znaor A, Soerjomataram I, Bray F. 2018. Global Cancer Observatory: Cancer Today. International Agency for Research on Cancer. https://gco.iarc.fr/today (accessed 7.1.19).
Jappinen, N., Felix, I., Lokka, E., Tyystjarvi, S., Pynttari, A., Lahtela, T., Gerke, H., Elima, K., Rantakari, P., Salmi, M., 2019. Fetal-derived macrophages dominate in adult mammary glands. Nat Commun 10, 281. doi:10.1038/s41467-018-08065-1
Jung, Y., Decker, A. M., Wang, J., Lee, E., Kana, L. A., Yumoto, K., Cackowski, F. C., Rhee, J., Carmeliet, P., Buttitta, L., Morgan, T. M., Taichman, R. S., 2016. Endogenous GAS6 and Mer receptor signaling regulate prostate cancer stem cells in bone marrow. Oncotarget 7, 25698-25711. doi:10.18632/oncotarget.8365
Kim, D., Choi, B.-H., Ryoo, I.-G., Kwak, M.-K., 2018. High NRF2 level mediates cancer stem cell-like properties of aldehyde dehydrogenase (ALDH)-high ovarian cancer cells: inhibitory role of all-trans retinoic acid in ALDH/NRF2 signaling. Cell Death Dis 9, 896. doi:10.1038/s41419-018-0903-4
Kipps, E., Tan, D. S. P., Kaye, S. B., 2013. Meeting the challenge of ascites in ovarian cancer: new avenues for therapy and research. Nat. Rev. Cancer 13, 273-282. doi:10.1038/nrc3432
Komohara, Y., Jinushi, M., Takeya, M., 2014. Clinical significance of macrophage heterogeneity in human malignant tumors. Cancer Sci. 105, 1-8. doi:10.1111/cas.12314
Kreso, A., Dick, J. E., 2014. Evolution of the cancer stem cell model. Cell Stem Cell 14, 275-291. doi:10.1016/j.stem.2014.02.006
Krist, L. F. G., Eestermans, I. L., Steenbergen, J. J. E., Hoefsmit, E. C. M., Cuesta, M. A., Meyer, S., Beelen, R. H. J., 1995. Cellular composition of milky spots in the human greater omentum: An immunochemical and ultrastructural study. The Anatomical Record 241, 163-174. doi:10.1002/ar.1092410204
Lahmar, Q., Keirsse, J., Laoui, D., Movahedi, K., Van Overmeire, E., van Ginderachter, J. A., 2016a. Tissue-resident versus monocyte-derived macrophages in the tumor microenvironment. Biochimica et Biophysica Acta (BBA)—Reviews on Cancer 1865, 23-34. doi:10.1016/j.bbcan.2015.06.009
Lahmar, Q., Keirsse, J., Laoui, D., Movahedi, K., Van Overmeire, E., van Ginderachter, J. A., 2016b. Tissue-resident versus monocyte-derived macrophages in the tumor microenvironment. Biochim Biophys Acta 1865, 23-34. doi:10.1016/j.bbcan.2015.06.009
Lee, C.-C., Lin, J.-C., Hwang, W.-L., Kuo, Y.-J., Chen, H.-K., Tai, S.-K., Lin, C.-C., Yang, M.-H., 2018. Macrophage-secreted interleukin-35 regulates cancer cell plasticity to facilitate metastatic colonization. Nat Commun 9, 3763. doi:10.1038/s41467-018-06268-0
Lengyel, E., 2010. Ovarian Cancer Development and Metastasis. Am. J. Pathol. 177 , 1053-1064. doi:10.2353/ajpath.2010.100105
Levina, V. V., Nolen, B., Su, Y., Godwin, A. K., Fishman, D., Liu, J., Mor, G., Maxwell, L. G., Herberman, R. B., Szczepanski, M. J., Szajnik, M. E., Gorelik, E., Lokshin, A. E., 2009. Biological significance of prolactin in gynecologic cancers. Cancer Res 69, 5226-5233. doi:10.1158/0008-5472.CAN-08-4652
Lim, H. Y., Lim, S. Y., Tan, C. K., Thiam, C. H., Goh, C. C., Carbajo, D., Chew, S. H. S., See, P., Chakarov, S., Wang, X. N., Lim, L. H., Johnson, L. A., Lum, J., Fong, C. Y., Bongso, A., Biswas, A., Goh, C., Evrard, M., Yeo, K. P., Basu, R., Wang, J. K., Tan, Y., Jain, R., Tikoo, S., Choong, C., Weninger, W., Poidinger, M., Stanley, E. R., Collin, M., Tan, N. S., Ng, L. G., Jackson, D. G., Ginhoux, F., Angeli, V., 2018. Hyaluronan Receptor LYVE-1-Expressing Macrophages Maintain Arterial Tone through Hyaluronan-Mediated Regulation of Smooth Muscle Cell Collagen. Immunity 49, 326-341.e7. doi:10.1016/j.immuni.2018.06.008
Love, M. I., Huber, W., Anders, S., 2014. Moderated estimation of fold change and dispersion for RNA-seq data with DESeq2. Genome Biol 15, 550. doi:10.1186/s13059-014-0550-8
Loyher, P.-L., Hamon, P., Laviron, M., Meghraoui-Kheddar, A., Goncalves, E., Deng, Z., Torstensson, S., Bercovici, N., de Chanville, C. B., Combadière, B., Geissmann, F., Savina, A., Combadière, C., Boissonnas, A., 2018. Macrophages of distinct origins contribute to tumor development in the lung. Journal of Experimental Medicine 411, jem.20180534-193. doi:10.1084/jem.20180534
Michela Lupia, U. C., 2017. Ovarian cancer stem cells: still an elusive entity? Mol. Cancer 16, 7. doi:10.1186/s12943-017-0638-3
Mossadegh-Keller, N., Gentek, R., Gimenez, G., Bigot, S., Mailfert, S., Sieweke, M. H., 2017. Developmental origin and maintenance of distinct testicular macrophage populations. J. Exp. Med. 214, 2829-2841. doi:10.1084/jem.20170829
Nieto, M. A., Huang, R. Y.-J., Jackson, R. A., Thiery, J. P., 2016. EMT: 2016. Cell 166 , 21-45. doi:10.1016/j.ce11.2016.06.028
Noy, R., Pollard, J. W., 2014. Tumor-Associated Macrophages: From Mechanisms to Therapy. Immunity 41, 49-61. doi:10.1016/j.immuni.2014.06.010
Ojalvo, L. S., Whittaker, C. A., Condeelis, J. S., Pollard, J. W., 2010. Gene Expression Analysis of Macrophages That Facilitate Tumor Invasion Supports a Role for Wnt-Signaling in Mediating Their Activity in Primary Mammary Tumors. J Immunol 184, 702-712. doi: 10.4049/j immuno1.0902360
Pearce, O. M. T., Delaine-Smith, R. M., Maniati, E., Nichols, S., Wang, J., Bohm, S., Rajeeve, V., Ullah, D., Chakravarty, P., Jones, R. R., Montfort, A., Dowe, T., Gribben, J., Jones, J. L., Kocher, H. M., Serody, J. S., Vincent, B. G., Connelly, J., Brenton, J. D., Chelala, C., Cutillas, P. R., Lockley, M., Bessant, C., Knight, M. M., Balkwill, F. R., 2018. Deconstruction of a Metastatic Tumor Microenvironment Reveals a Common Matrix Response in Human Cancers. Cancer Discov 8, 304-319. doi:10.1158/2159-8290.CD-17-0284
Pollard, J. W., 2009. Trophic macrophages in development and disease. Nat Rev Immunol 9, 259-270. doi:10.1038/nri2528
Raggi, C., Mousa, H. S., Correnti, M., Sica, A., Invernizzi, P., 2015. Cancer stem cells and tumor-associated macrophages: a roadmap for multitargeting strategies. Oncogene 35 , 671-682. doi:10.1038/onc.2015.132
Rangel-Moreno, J., Moyron-Quiroz, J. E., Carragher, D. M., Kusser, K., Hartson, L., Moquin, A., Randall, T. D., 2009. Omental Milky Spots Develop in the Absence of Lymphoid Tissue-Inducer Cells and Support B and T Cell Responses to Peritoneal Antigens. Immunity 30, 731-743. doi:10.1016/j.immuni.2009.03.014
Roby, K. F., Taylor, C. C., Sweetwood, J. P., Cheng, Y., Pace, J. L., Tawfik, O., Persons, D. L., Smith, P. G., Terranova, P. F., 2000. Development of a syngeneic mouse model for events related to ovarian cancer. Carcinogenesis 21, 585-591. doi:10.1093/carcin/21.4.585
Rosas, M., Davies, L. C., Giles, P. J., Liao, C.-T., Kharfan, B., Stone, T. C., O'Donnell, V. B., Fraser, D. J., Jones, S. A., Taylor, P. R., 2014. The transcription factor Gata6 links tissue macrophage phenotype and proliferative renewal. Science 344, 645-648. doi:10.1126/science.1251414
Schreiber, H. A., Loschko, J., Karssemeijer, R. A., Escolano, A., Meredith, M. M., Mucida, D., Guermonprez, P., Nussenzweig, M. C., 2013. Intestinal monocytes and macrophages are required for T cell polarization in response to Citrobacter rodentium. J. Exp. Med. 210, 2025-2039. doi:10.1084/jem.20130903
Schulz, C., Perdiguero, E. G., Chorro, L., Szabo-Rogers, H., Cagnard, N., Kierdorf, K., Prinz, M., Wu, B., Jacobsen, S. E. W., Pollard, J. W., Frampton, J., Liu, K. J., Geissmann, F., 2012. A Lineage of Myeloid Cells Independent of Myb and Hematopoietic Stem Cells. Science 336, 86-90. doi:10.1126/science.1219179
Scott, C. L., Zheng, F., De Baetselier, P., Martens, L., Saeys, Y., De Prijck, S., Lippens, S., Abels, C., Schoonooghe, S., Raes, G., Devoogdt, N., Lambrecht, B. N., Beschin, A., Guilliams, M., 2016. Bone marrow-derived monocytes give rise to self-renewing and fully differentiated Kupffer cells. Nat Commun 7, 10321. doi:10.1038/ncomms10321
Sehouli, J., Senyuva, F., Fotopoulou, C., Neumann, U., Denkert, C., Werner, L., Gülten, O. Ö., 2009. Intra-abdominal tumor dissemination pattern and surgical outcome in 214 patients with primary ovarian cancer. J Surg Oncol 99, 424-427. doi:10.1002/jso.21288
Solar, P., Feldman, L., Jeong, J. Y., Busingye, J. R., Sytkowski, A. J., 2008. Erythropoietin treatment of human ovarian cancer cells results in enhanced signaling and a paclitaxel-resistant phenotype. Int. J. Cancer 122, 281-288. doi:10.1002/ijc.23071
Soucie, E. L., Weng, Z., Geirsdottir, L., Molawi, K., Maurizio, J., Fenouil, R., Mossadegh-Keller, N., Gimenez, G., VanHille, L., Beniazza, M., Favret, J., Berruyer, C., Perrin, P., Hacohen, N., Andrau, J. C., Ferrier, P., Dubreuil, P., Sidow, A., Sieweke, M. H., 2016. Lineage-specific enhancers activate self-renewal genes in macrophages and embryonic stem cells. Science 351, aad5510. doi:10.1126/science.aad5510
Torchilin, V. P., Levchenko, T. S., Lukyanov, A. N., Khaw, B. A., Klibanov, A. L., Rammohan, R., Samokhin, G. P., Whiteman, K. R., 2001. p-Nitrophenylcarbonyl-PEG-PE-liposomes: fast and simple attachment of specific ligands, including monoclonal antibodies, to distal ends of PEG chains via p-nitrophenylcarbonyl groups. Biochim Biophys Acta 1511, 397-411.
Wang, Y., Zong, X., Mitra, S., Mitra, A. K., Matei, D., Nephew, K. P., 2018. IL-6 mediates platinum-induced enrichment of ovarian cancer stem cells. JCI Insight 3, 87. doi:10.1172/jci.insight.122360
Wehrens, R., Buydens, L. M. C., 2007. Self- and Super-organizing Maps in R: The kohonenPackage. Journal of Statistical Software 21. doi:10.18637/jss.v021105
Wehrens, R., Kruisselbrink, J., 2018. Flexible Self-Organizing Maps in kohonen3.0. Journal of Statistical Software 87. doi:10.18637/jss.v087107
Yan, W., Cao, Q. J., Arenas, R. B., Bentley, B., Shao, R., 2010. GATA3 inhibits breast cancer metastasis through the reversal of epithelial-mesenchymal transition. J. Biol. Chem. 285, 14042-14051. doi:10.1074/jbc.M110.105262
Yang, M., McKay, D., Pollard, J. W., Lewis, C. E., 2018. Diverse Functions of Macrophages in Different Tumor Microenvironments. Cancer Res 78, 5492-5503. doi:10.1158/0008-5472.CAN-18-1367
Yang, Z., He, L., Lin, K., Zhang, Y., Deng, A., Liang, Y., Li, C., Wen, T., 2017. The KMT1A-GATA3-STAT3 Circuit Is a Novel Self-Renewal Signaling ofHuman Bladder Cancer Stem Cells. Clin. Cancer Res. 23, 6673-6685. doi:10.1158/1078-0432.CCR-17-0882
Yona, S., Kim, K.-W., Wolf, Y., Mildner, A., Varol, D., Breker, M., Strauss-Ayali, D., Viukov, S., Guilliams, M., Misharin, A., Hume, D. A., Perlman, H., Malissen, B., Zelzer, E., Jung, S., 2013. Fate mapping reveals origins and dynamics of monocytes and tissue macrophages under homeostasis. Immunity 38, 79-91. doi:10.1016/j.immuni.2012.12.001
Yu, G., Wang, L.-G., Han, Y., He, Q.-Y., 2012. clusterProfiler: an R package for comparing biological themes among gene clusters. OMICS 16, 284-287. doi:10.1089/omi.2011.0118
Zhu, Y., Herndon, J. M., Sojka, D. K., Kim, K.-W., Knolhoff, B. L., Zuo, C., Cullinan, D. R., Luo, J., Bearden, A. R., Lavine, K. J., Yokoyama, W. M., Hawkins, W. G., Fields, R. C., Randolph, G. J., DeNardo, D. G., 2017. Tissue-Resident Macrophages in Pancreatic Ductal Adenocarcinoma Originate from Embryonic Hematopoiesis and Promote Tumor Progression. Immunity 47, 323-338.e6. doi:10.1016/j.immuni.2017.07.014

1. A method of treating a cancer or preventing resistance to an immune checkpoint therapy, radiotherapy or chemotherapy of the cancer in a subject in need thereof;
the method comprising administering to the subject a therapeutically effective amount of an agent capable of depleting the population of CD163+ Tim4+ macrophages and CD163− Tim4+ macrophages in the subject's tumor;
wherein the agent is an antibody having binding affinity for Tim4.

2-22. (canceled)

23. The method of claim 1, wherein the cancer is selected from the group consisting of ovarian cancer, pancreatic cancer and breast cancer.

24. The method of claim 1, wherein the cancer is ovarian cancer.

25. The method of claim 1, wherein the cancer is resistant to immune checkpoint therapy, radiotherapy or chemotherapy.

26. The method of claim 1, wherein the antibody binds to the extracellular domain of Tim4.

27. The method of claim 1, wherein the antibody is an antibody-drug conjugate.

28. The method of claim 1, wherein the antibody is conjugated to a cytotoxic moiety.

29. The antibody of claim 1, wherein the antibody is conjugated to doxorubicin and/or duocarmycin.

30. The method of claim 1, wherein the antibody mediates antibody-dependent cell-mediated cytotoxicity.

31. The method according to claim 1, further comprising administering to the subject a therapeutically effective amount of a second agent capable of depleting the populations of CD163+ Tim4+ macrophages and CD163+ Tim4− macrophages in the subject's tumor;
wherein the second agent is an antibody having binding affinity for CD163.

32. The method according to claim 1, further comprising administering to the subject a therapeutically effective amount of a second agent capable of depleting the population of CD163+ Tim4+ and CD163+ Tim4− macrophages in the subject's tumor;
wherein the second agent is an antibody having binding affinity for CD163;
the method thereby leading to depletion of CD163+ Tim4+ macrophages, CD163− Tim4+ macrophages and CD163+ Tim4− macrophages in the subject's tumor.

33. The method of claim 1, wherein the antibody is a multispecific antibody comprising a first antigen binding site directed against Tim4 and a second antigen binding site directed against CD163.

34. The method of claim 1, wherein the antibody is a multispecific antibody comprising a first antigen binding site directed against Tim4 and a second antigen binding site directed against CD163; wherein the method is for depletion of CD163+ Tim4+ macrophages, CD163− Tim4+ macrophages and CD163+ Tim4− macrophages in the subject's tumor.

35. A method of treating a cancer or preventing resistance to immune checkpoint therapy, radiotherapy or chemotherapy of a cancer in a subject in need thereof;
the method comprising administering to the subject a combination comprising an antibody having binding affinity for Tim4 and an antibody having binding affinity for CD163.

36. The method of claim 35, wherein the cancer is selected from the group consisting of ovarian cancer, pancreatic cancer and breast cancer.

37. The method according to claim 35, wherein the method leads to the depletion of CD163+ Tim4+ macrophages, CD163− Tim4+ macrophages and CD163+ Tim4− macrophages in the subject's tumor.

38. The method according to claim 34, wherein the antibody having binding affinity for CD163 and/or the antibody having binding affinity for Tim4 is an antibody-drug conjugate.

39. A kit of parts comprising:
a first container comprising an antibody having binding affinity for Tim4; and
a second container comprising an antibody having binding affinity for CD163.

40. The kit of parts according to claim 39, wherein the antibody having binding affinity for CD163 and antibody having binding affinity for Tim4 are antibody-drug conjugates.

41. The kit according to claim 39, wherein the antibody having binding affinity for CD163 and/or the antibody having binding affinity for Tim4 is conjugated to doxorubicin and/or duocarmycin.


SEQUENCE LISTING (2 sequences)

SEQ ID NO: 1 - protein, 1156 aa, Homo sapiens
MSKLRMVLLEDSGSADFRRHFVNLSPFTITVVLLLSACFVTSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAIWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSENSHESADFSAAELISVSKFLPISGMEKEAILSHTEKENGNL

SEQ ID NO: 2 - protein, 378 aa, Homo sapiens
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTL